(19) United States
(12) Patent Application Publication
(10) Pub. No.: US 2011/0245171 A1
(43) Pub. Date: Oct. 6, 2011

(54) REHYDRATION COMPOSITIONS COMPRISING EPIDERMAL GROWTH FACTOR (EGF)

(76) Inventors: James A. Hardin, Calgary (CA); Merle E. Olson, Calgary (CA); D. Grant Gall, Calgary (CA); Loretta Gall, Calgary (CA); Andre G. Buret, Calgary (CA)

(21) Appl. No.: 11/576,303

(22) PCT Filed: Sep. 29, 2005
(86) PCT No.: PCT/CA05/01488

§ 371 Date: Apr. 18, 2011

Related U.S. Application Data
(60) Provisional application No. 60/614,428, filed on Sep. 30, 2004.
(60) Provisional application No. 60/636,855, filed on Dec. 20, 2004.

(51) Int. Cl. A61K 38/18; A61P 1/12

(52) U.S. Cl. 514/8.9; 514/9.6

(57) ABSTRACT

The invention comprises 1) an oral composition, including an oral rehydration composition or solution, comprising an effective amount of a compound selected from epidermal growth factor (EGF) and agonists to the epidermal growth factor receptor, 2) a kit comprising an oral rehydration composition containing an effective amount of a compound selected from epidermal growth factor and agonists to the epidermal growth factor receptor, and 3) methods for the treatment and prevention of dehydration and diarrhea, and enhancing intestinal healing, reducing bacterial colonization, reducing the incidence of weight loss, increasing food uptake, enhancing rehydration, and enhancing mucosal healing in an animal having diarrhea.

### FIELD OF THE INVENTION

This invention relates generally to compositions, kits and methods for treating and preventing dehydration and diarrhea.

### BACKGROUND

Oral rehydration therapy is administered to treat dehydration. Dehydration occurs when water losses exceed water intake. This can be due to excessive fluid loss through perspiration during exercise or high temperature exposure, but clinically is most often observed during gastrointestinal disturbances. These disorders may be related to secretion, i.e. secretory diarrhea due to release of a toxin by the pathogenic agent that produces a profuse watery diarrhea like that seen in cholera, or malabsorption (decrease in absorptive function such that a increased osmotic load in the lumen of the gut pulls water from the body leading to diarrhea or inadequate absorption of water and nutrients i.e. certain bacterial infections or malabsorptive conditions such as short bowel syndrome). Current oral rehydration therapy is based upon the active, coupled uptake of Na⁺ and nutrients in the small intestine, and the subsequent influx of water that follows the movement of absorbed solute (55).

The absorption of many water-soluble nutrients across the intestinal epithelium is driven by the electrochemical gradient for Na⁺ across the apical membrane generated by Na⁺K⁺ATPape in the basolateral membrane (1). Glucose and galactose are actively transported across the apical membrane by the Na⁺-dependent glucose transporter (SGLT1) (87). Fructose, in contrast, is transported across the apical membrane by non-Na⁺-dependent facilitated diffusion mediated by the glucose transporter GLUT5. Sugar molecules then exit the basolateral membrane of the enterocyte by facilitated diffusion via GLUT2 (1). Water passively follows the movement of absorbed solute (24;44). The mechanism/route by which water actually crosses the intestinal epithelium is not clear. The most likely pathway involves the passive flow of water through the tight junctions that form the connections between cells of the intestinal epithelium (the “paracellular” route) (39;44;52). However, water channel proteins have been identified on intestinal epithelial cells suggesting a possible role for these proteins in “transcellular” water movement through cells (51). Finally, a number of reports suggest that sodium-coupled nutrient transporters like SGLT1 may cotransport water by forming a hydration shell around the transported solute (49;50).

Studies have demonstrated a role for mucosal epidermal growth factor (EGF) in the acute up-regulation of jejunal nutrient (9;38;40;54;61;71;72;76;82) and water (61;71;72) transport. EGF rapidly increases glucose and amino acid transport associated with an increase in brush border surface area that occurs within minutes (34;38), suggesting a rapid insertion of apical membrane from a preformed intracellular pool (34). Transforming growth factor alpha (TGFα), a related peptide that binds to the EGF receptor, had no effect on glucose transport or apical surface area indicating the receptor interaction mediating this effect is specific for EGF (36) although some studies have reported an increase in small intestinal absorption following TGFα administration (71). Further studies demonstrated mucosal EGF increased the Vmax for glucose and proline uptake into brush border membrane vesicles (BBMV) (38). The EGF-induced increase in the Vmax for BBMV glucose uptake is associated with a significant increase in BBMV SGLT1 content (26). Recent studies have identified SGLT1 protein in enterocyte microsomal membranes (25). EGF treatment resulted in a decrease in SGLT1 content in the microsomal fraction and a concomitant increase in brush border SGLT1 content. Furthermore, SGLT1 immunofluorescence extended further down the villus in EGF treated tissue suggesting a recruitment of additional enterocytes into the glucose-transporting compartment (25). EGF has also been reported to increase jejunal glutamine, alanine galactose and glycine transport (73), and ileal electroneutral NaCl absorption and brush-border Na⁺/H⁺ exchange (29). These findings suggest EGF acutely stimulates a general increase in nutrient transport via the acute insertion of a membrane pool enriched in nutrient transporters.

EGF also exhibits potent anti-infective properties. Studies have demonstrated the ability of EGF to significantly reduce the colonization of mucosal surfaces by a wide variety of pathogenic organisms (15). In vitro, EGF has been shown to inhibit epithelial bacterial translocation and invasion by pathogenic strains of Escherichia coli (12-14) and Salmonella typhimurium (12-14) and reduce epithelial colonization by the protozoan Cryptosporidium parvum (18;21;22). Furthermore, in a model of enteropathogenic E. coli infection, EGF treatment significantly inhibited bacterial colonization and reduced epithelial damage and prevented the diarrhea and reduced weight gain associated with the disease (12-14;16;20). In a model of C. parvum infection, EGF decreased fecal oocyte passage (21). In a model of giardiasis EGF treatment inhibited trophozoite colonization and enhanced intestinal function compared to untreated animals (37). Importantly, EGF has no direct bactericidal (20;30;31) or anti-protozoal (18;21) effect and thus avoids the problems associated with antibiotic resistance.

Dehydration is commonly associated with diarrheal disease. Secretory diarrhea involves the stimulation of active Cl⁻ secretion, which then induces the flux of water into the intestinal lumen. In these clinical states absorptive mechanisms remain intact and thus administration of oral rehydration solutions (ORS) containing Na⁺, glucose, electrolytes and water are able to counteract the water loss due to Cl⁻ secretion. Infectious diseases such as cholera, salmonella and many forms of traveler's diarrhea induce a secretory diarrhea (60). Conversely, malabsorptive osmotic diarrhea results from a loss of absorptive function in the intestine. Infections caused by Yersinia enterocolitica (58;59), Giardia sp. (11;17;79), Cryptosporidium (22) and rotavirus (60) have been shown to produce a malabsorptive diarrhea. Malabsorption often results from a loss of absorptive surface area. Studies have demonstrated a diffuse shortening of the brush border in some malabsorptive osmotic diarrheal diseases, i.e. Yersinia, enteropathogenic E. coli (20), and giardia infections (17;19;60). Malabsorbed nutrients in the intestinal lumen lead to a large osmotic load and subsequent flux of water into the lumen. Due to the dependence of conventional oral rehydration therapy on absorptive function, it is less effective in malabsorptive diarrheal states where absorptive function is impaired (5;74). Furthermore, although oral rehydration solutions stimulate the intestinal absorption of fluid and electrolytes from isotonic luminal contents, they do not aid in the reabsorption of fluid secreted by the intestine, and thus do not appreciably lessen the severity of diarrhea (5;56). In addition, current ORSs do not facilitate bowel repair (56;70).

Investigational research in the area of oral rehydration has focused on finding an optimal balance of nutrients and electrolytes (27;42), exploring the relative effectiveness of various nutrient sources (4;5;53;86), and supplementing oral rehydration with the addition or use of functional nutrients and agents designed to increase absorptive function and/or reduce the duration and frequency of diarrhea or enhance intestinal repair (2;8;67;68;70;74). Glutamine (23;43;47;68;70), alanine (69;74), glycine (6;83), zinc (2;7;8;70), copper (63), soluble fiber (68), gum arabic (85), nitric oxide (3), tormentil root extract (78), and probiotics (67;70) are some of the functional nutrients and agents that have been examined for their potential in supplementing oral rehydration solutions.

### SUMMARY OF THE INVENTION

The invention relates to oral compositions comprising EGF, EGF receptor agonists or pharmaceutically acceptable salt forms thereof. The oral composition may be an aqueous oral rehydration composition, wherein the oral rehydration composition may comprise EGF, EGF receptor agonists or pharmaceutically acceptable salt forms thereof, in combination with water and a component or components that will actively enhance the absorption of water from the lumen of the gastrointestinal tract. For example, the oral rehydration composition may comprise at least one of the following solutes (i) salts, which dissociate into their respective charged ions/electrolytes in solution for rehydration, (ii) carbohydrates, or (iii) other alternative sodium-coupled nutrient or a source of sodium-coupled nutrients (i.e. amino acids, or a source of amino acids, for example peptides and polypeptides, or short-chain fatty acids or a source of non-digestible carbohydrate which can be metabolized to short-chain fatty acids by bacterial fermentation in the gut). For example, the oral rehydration composition may be Gatorade™ or sports drinks supplemented with EGF. Normally, the aqueous oral rehydration composition would include some form of carbohydrate, or other alternative sodium-coupled nutrient or a source of sodium-coupled nutrients (i.e. amino acids, or a source of amino acids, for example peptides and polypeptides, or short-chain fatty acids or a source of non-digestible carbohydrate which can be metabolized to short-chain fatty acids by bacterial fermentation in the gut). The aqueous rehydration composition may also include flavourings, preservatives, and colouring agents. Alternatively, the oral rehydration composition is an oral rehydration solution (ORS). The oral rehydration solution of the present invention may comprise EGF, EGF receptor agonists or pharmaceutically acceptable salt forms thereof in combination with components of ORS, for example water, sodium, potassium, chloride, a source of base and a source of carbohydrates or amino acids. An example of a source of base is citrate, which is metabolized to bicarbonate, the base in the blood that helps maintain acid-base balance. While citrate is an excellent source of base, any base routinely incorporated into rehydration solutions may be used in the practice of the current invention.

Epidermal growth factor refers to any epidermal growth factor, or a variant, analog, fragment, or derivative thereof. Epidermal growth factor receptor agonists may include but are not limited to EGF, transforming growth factor alpha, amphiregulin, heparin binding EGF, and epiregulin.

The present invention provides an oral composition comprising an epidermal growth factor (EGF), an epidermal growth factor receptor agonist, or a pharmaceutically acceptable salt form thereof. Although the invention describes oral compositions, the compositions can also be delivered enterally, for example by nasogastric tube, to achieve the same result. This is known to those skilled in the art.

An aspect of the invention is to provide use of an oral composition comprising an epidermal growth factor (EGF), an epidermal growth factor receptor agonist, or a pharmaceutically acceptable salt form thereof for treating diarrhea, reducing the severity of diarrhea, reducing the duration of diarrhea, promoting intestinal healing of intestinal damage associated with diarrhea, treating dehydration associated with diarrhea, reducing bacterial colonization in an established diarrhea infection, reducing weight loss in an animal having diarrhea, increasing food uptake in an animal having diarrhea, enhancing rehydration in an animal having diarrhea, reducing water content in fecal matter in an animal having diarrhea, increasing villus height in an animal having diarrhea, improving mucosal healing in an animal having diarrhea, and enhancing mucosal wet weight in an animal having diarrhea. For example, the diarrhea can be infectious malabsorptive diarrhea, neonatal diarrhea, or secretory diarrhea. The composition can be an ORS. The composition can be a solution, suspension, colloid, concentrate, powder, granules, tablets, pressed tablets or capsules.

The composition can comprise from about 1 ng/kg/day to about 10 mg/kg/day of epidermal growth factor, epidermal growth factor receptor agonist or pharmaceutically acceptable salt forms thereof. The composition can comprise from about 0.1 μg/kg/day to about 1 mg/kg/day of epidermal growth factor, epidermal growth factor receptor agonist or pharmaceutically acceptable salt forms thereof. Alternatively, the composition can comprise from about 1 μg/kg/day to about 1 mg/kg/day of epidermal growth factor, epidermal growth factor receptor agonist or pharmaceutically acceptable salt forms thereof. Finally, the composition can comprise from about 1 μg/kg/day to about 0.1 mg/kg/day of epidermal growth factor, epidermal growth factor receptor agonist or pharmaceutically acceptable salt forms thereof.

Another aspect of the present invention is to provide an aqueous oral rehydration composition comprising (i) an epidermal growth factor (EGF), an epidermal growth factor receptor agonist, or a pharmaceutically acceptable salt form thereof; (ii) a carbohydrate, and (iii) at least one solute selected from the group consisting of salts and an alternative sodium-coupled nutrient or a source of a sodium-coupled nutrients. The sodium-coupled nutrient or the source of sodium-coupled nutrients is selected from the group consisting of amino acids, a source of amino acids, peptides, polypeptides, short-chain fatty acids and a source of non-digestible carbohydrate which can be metabolized to short-chain fatty acids by bacterial fermentation in the gut. The composition can comprise from about 100 picograms to about 1 milligram of epidermal growth factor or epidermal growth factor receptor agonist per milliliter, or from about 1 nanogram to about 100 micrograms of epidermal growth factor or epidermal growth factor receptor agonist per milliliter, or from 10 nanograms to about 10 micrograms of epidermal growth factor or epidermal growth factor receptor agonist per milliliter. The rehydration composition can be an oral rehydration solution (ORS).

The ORS can comprise sodium, potassium, chloride, a source of base, and a carbohydrate or a sodium-coupled nutrient or a source of a sodium-coupled nutrients. The sodium can be present from about 30 mEq/L to about 95 mEq/L, the potassium can be present from about 10 mEq/L to about 30 mEq/L, the carbohydrate can be present at less than about 5% w/w, (d) the source of base is present from about 10 mEq/L to about 40 mEq/L, and the chloride can be present from about 30 mEq/L to about 80 mEq/L. Alternatively, the sodium can be present from about 30 mEq/L to about 70 mEq/L, the potassium can be present from about 15 mEq/L to about 25 mEq/L, the carbohydrate can be present at less than about 3% w/w, the source of base can be present from about 20 mEq/L to about 40 mEq/L, and the chloride can be present from about 30 mEq/L to about 75 mEq/L. Alternatively, the sodium can be present from about 40 mEq/L to about 60 mEq/L, the potassium can be present from about 15 mEq/L to about 25 mEq/L, the carbohydrate can be present from about 2% to about 3% w/w, the source of base can be present from about 25 mEq/L to about 35 mEq/L, and the chloride can be present from about 30 mEq/L to about 70 mEq/L. The source of base can be selected from the group consisting of potassium citrate, sodium citrate, citric acid and mixtures thereof, the carbohydrate can be selected from the group consisting of glucose, dextrose, fructooliogosaccharides, fructose polymers, glucose polymers, corn syrup, high fructose corn syrup, sucrose, maltodextrin, rice, rice flour and mixtures thereof, the sodium can be selected from the group consisting of sodium chloride, sodium citrate, sodium bicarbonate, sodium carbonate, sodium hydroxide, and mixtures thereof, the potassium can be selected from the group consisting of potassium citrate, potassium chloride, potassium bicarbonate, potassium carbonate, potassium hydroxide and mixtures thereof, and the chloride can be selected from the group consisting of potassium chloride, sodium chloride, zinc chloride and mixtures thereof. Finally, the sodium-coupled nutrient or source of the sodium-coupled nutrients can be selected from the group consisting of amino acids, a source of amino acids, peptides, polypeptides, short-chain fatty acids and a source of non-digestible carbohydrate which can be metabolized to short-chain fatty acids by bacterial fermentation in the gut.

The oral rehydration composition can further comprise zinc, glutamine, indigestible oligosaccharide, amidine derivatives, an additional pharmaceutically active ingredient, an absorptive component, and/or a glycolipid. The composition can be frozen or in the form of a gel. The composition can further comprise a sweetener, flavouring, preservatives, an excipient, a diluent, or an adjuvant.

Another aspect of the invention is to provide a kit comprising (a) a therapeutic amount of an epidermal growth factor, an epidermal growth factor receptor agonist, or a pharmaceutically acceptable salt form thereof, (b) at least one solute selected from the group consisting of sodium, potassium, chloride, a source of base, a carbohydrate, an alternative sodium-coupled nutrient and a source of sodium-coupled nutrients, and (c) instructions.

Another aspect of the invention is to provide a method of manufacturing an oral composition comprising providing an epidermal growth factor, and epidermal growth factor receptor agonist, or a pharmaceutically acceptable salt form thereof in the composition and manufacturing the oral composition. The oral composition can be an ORS.

Another aspect of the invention is to provide a method of manufacturing a kit comprising providing (a) a therapeutic amount of an epidermal growth factor, and epidermal growth factor receptor agonist, or a pharmaceutically acceptable salt form thereof, (b) at least one solute selected from the group consisting of sodium, potassium, chloride, a source of base, a carbohydrate, an alternative sodium-coupled nutrient and a source of sodium-coupled nutrients, and (c) instructions.

Another aspect of the invention is to provide a method of manufacturing a kit comprising providing (a) a therapeutic amount of an epidermal growth factor, (b) at least one solute selected from the group consisting of sodium, potassium, chloride, a source of base, a carbohydrate, sodium-coupled nutrient and a source of sodium-coupled nutrients, and (c) instructions.

Another aspect of the invention is to provide a method for treating diarrhea, reducing the severity of diarrhea, reducing the duration of diarrhea, promoting intestinal healing, treating dehydration, reducing bacterial colonization, increasing food uptake, enhancing rehydration, reducing water content in fecal matter, increasing villus height, improving mucosal healing, and enhancing mucosal wet weight in an animal having diarrhea, comprising administering to an animal having diarrhea an effective amount of an epidermal growth factor, an epidermal growth factor receptor agonist, or a pharmaceutically acceptable salt form thereof. For example, the diarrhea can be infectious malabsorptive diarrhea, neonatal diarrhea, or secretory diarrhea. The animal can be selected from the group consisting of human, dog, cat, cow, horse, pig, goat, sheep, rabbits, mink, llamas, alpacas, elk, bison, fish and poultry. The epidermal growth factor can be in any one of the compositions described above. The method can further be used to reduce weight loss in an animal having diarrhea.

Another aspect of the invention is to provide a method of treating diarrhea in an animal having a condition selected from the group consisting of recovery from gastrointestinal surgery, gastrointestinal resection, small intestinal transplant, post surgical trauma, short bowel syndrome, burns, oral mucositis, AIDS, inflammatory diseases, Crohn's disease, Ulcerative colitis, celiac disease, necrotizing enterocolitis, gut prematurity, bone marrow transplants, intestinal damage due to chemotherapy or radiation therapy, sepsis, intestinal infections and subjects requiring total parenteral nutrition (TPN) comprising administering the composition described above.

Another aspect of the invention is to provide a unit dose of epidermal growth factor, epidermal growth factor receptor agonist, or pharmaceutically acceptable salt form thereof for use in a mixture with an oral rehydration solution.

Another aspect of the invention is to provide a composition delivered enterally, comprising epidermal growth factor, epidermal growth factor receptor agonist, or pharmaceutically acceptable salt form thereof. The enteral composition has use in any of the methods or uses described above.

The supplementation of oral rehydration solutions with EGF, an EGF receptor agonist or a salt form thereof will provide significant clinical benefit. As described above, oral EGF rapidly enhances glucose transport (38;61) and microvillus surface area (34) in the small intestine. EGF has been shown to increase enterocyte glucose transport in the presence of cholera toxin (54) and reverse the defect in glucose transport observed following administration of the somatostatin analogue octreotide (48;75). Stimulating glucose transport and absorptive surface area will increase the efficacy of ORS in both secretory and malabsorptive osmotic diarrheal states. Furthermore, the anti-infective properties of EGF will decrease mucosal colonization by pathogens and act to limit the severity of the disease (15). Finally, EGF has been shown to play a role in maintaining mucosal integrity and enhancing mucosal wound repair (33;64;66;84). These properties would provide additional benefit in the context of an oral rehydration solution.

### BRIEF DESCRIPTION OF THE FIGURES

FIG. 1. Jejunal 3-O-methyl glucose transport in tissue obtained from control rabbits, rabbits infected with attaching-effacing E. coli (RDEC-1), and infected rabbits with subsequent EGF administration. Values are mean±SEM (n=5 for controls, n=4 for the infected groups).

FIG. 2. Mean fecal scores for EGF-treated piglets (I.EGF.T.) and Untreated piglets (I.UT.) infected with E. coli K-88 (1×10¹⁰). EGF treatment was initiated on day-1, and animals were infected on day 0. Values are mean±SEM (n=6 for controls, n=5 for test group).

FIG. 3. Mean fecal scores for EGF-treated piglets (EGF-treated) and Untreated piglets (Untreated) with spontaneous neonatal diarrhea. EGF treatment was initiated on day 0 (t=0) and continued for 24 hrs (t=24 hr). Values are mean±SEM.*P<0.05 vs. EGF-treated group at t=0. (n=5 for controls, n=5 for test group).

FIG. 4. Effect of infection with attaching-effacing E. coli (RDEC-1) and treatment with ORS or ORS+EGF on percent weight gain. Values are mean±SEM.*=P<0.05 infected ORS versus Controls.

FIG. 5. Food intake in controls and animals infected with attaching-effacing E. coli (RDEC-1) treated with ORS or ORS+EGF. Values are mean±SEM.*=P<0.05 Infected ORS+EGF compared to both infected ORS and controls.

FIG. 6. Fecal scoring in controls and animals infected with attaching-effacing E. coli (RDEC-1) treated with ORS or ORS+EGF. Values are mean±SEM.*=P<0.01 Infected ORS versus controls.

FIG. 7. Hematocrits on day 5 in controls and animals infected with attaching-effacing E. coli (RDEC-1) treated with ORS or ORS+EGF. Values are mean±SEM.*=P<0.05 Infected ORS versus controls.

FIG. 8. Fecal water content of fecal samples collected at autopsy from controls and animals infected with attaching-effacing E. coli (RDEC-1) treated with ORS or ORS+EGF. Values are mean±SEM.*=P<0.001 Infected ORS versus controls, +=P<0.001 Infected ORS compared to Infected ORS+EGF.

FIG. 9. Villus height in jejunal tissue from controls and animals infected with attaching-effacing E. coli (RDEC-1) treated with ORS or ORS+EGF. Values are mean±SEM.*=P<0.01 Infected ORS versus controls. +=P<0.05 Infected ORS compared to Infected ORS+EGF.

FIG. 10. Jejunal mucosal wet weight in control animals and animals infected with attaching-effacing E. coli (RDEC-1) treated with ORS or ORS+EGF. Values are mean±SEM.*=P<0.05 Infected ORS compared to Infected ORS+EGF.

FIG. 11. Jejunal sucrase activity in control animals and animals infected with attaching-effacing E. coli (RDEC-1) treated with ORS or ORS+EGF. Values are mean±SEM.*=P<0.001 compared to control.

FIG. 12. Jejunal maltase activity in control animals and animals infected with attaching-effacing E. coli (RDEC-1) treated with ORS or ORS+EGF. Values are mean±SEM.*=P<0.05 compared to control. +=P<0.001 compared to control.

FIG. 13. Jejunal bacterial counts expressed as CFU's/cm jejunum in control animals and animals infected with attaching-effacing E. coli (RDEC-1) treated with ORS or ORS+EGF. Values are mean SEM.*=P<0.01 compared to control.

### DESCRIPTION OF THE INVENTION

Prior to setting forth the invention, it may be helpful to an understanding thereof to define certain terms that will be used hereinafter.

“Dehydration” as used herein means a condition resulting from excessive loss of body fluid that occurs when output of fluid exceeds fluid intake. This may result from fluid deprivation, excessive loss of fluid, reduction in total quantity of electrolytes, or injection of hypertonic solutions.

“Diarrhea” as used herein means a gastrointestinal condition characterized by the frequent passage of abnormally watery bowel movements.

“EGF receptor agonist” as used herein means any molecule which will produce a biochemical effect when bound to any of the erbB (1-4) receptors, particularly the erbB1 receptor, such that any or all of the following effects occur: intestinal glucose transport is increased, the apical surface of the enterocytes (cells lining lumen of the small intestine) are altered, the colonization and translocation of pathogenic organisms across mucosal surfaces is inhibited, and gut maturation is induced. For example, the molecule may be: an epidermal growth factor, an antibody, small molecule, protein, peptide, peptidic analogue, or peptidomimetic.

“Epidermal growth factor” or EGF as used herein refers to any epidermal growth factor, or a variant, analog, fragment, or derivative thereof. For example, EGF as used herein may be a 53-amino acid protein known to be synthesized in the duodenum and salivary glands of normal humans, and expressed in human breast milk. The amino acid sequence of human EGF is:

(SEQ ID NO: 1)
Asn Ser Asp Ser Glu Cys Pro Leu Ser His Asp Gly
Tyr Cys Leu His Asp Gly Val Cys Met Tyr Ile Glu
Ala Leu Asp Lys Tyr Ala Cys Asn Cys Val Val Gly
Tyr Ile Gly Glu Arg Cys Gln Tyr Arg Asp Leu Lys
Trp Trp Glu Leu Arg. (10)

The protein used in the experiments described herein had the foregoing sequence. Non-human EGF sequences which act as EGF in humans are also contemplated. Species variants of EGF are thus also included, such as described for mouse, rat and pig (45;57;62;77), or bovine EGF as cited in U.S. patent application 20030059802, or so-called supra-agonistic chimeras of different EGF receptor ligands (46). This definition also refers to a polypeptide having substantially the same sequence and activity as purified native epidermal growth factor. This includes recombinantly and chemically synthesized peptides or proteins. This term also refers to proteins varying from the native sequence by substitution with other amino acids or deletion of one or more amino acids, as long as the EGF biological activity is substantially preserved. The definition also includes fragments, peptidic analogues, and peptidomimetics of EGF as long as the EGF biological activity is substantially preserved. EGF biological activity can be screened by a receptor binding assay, and confirmed using any of the methods indicated above in connection with receptor agonists. Thus, for example, a human EGF protein in which the methionine (Met) at position 21 is replaced with isoleucine (Ile) falls within the scope of “EGF.” Such a protein is denoted hEGF-I₂₁ generally, and is generally denoted rhEGF-I₂₁ if prepared recombinantly (chemically synthesized hEGF is included in the term “hEGF”). Similarly, hEGF having the Asp at position 11 replaced with Glu is generally denoted hEGF-E₁₁. Some EGF proteins truncated near the carboxy terminal retain their biological activity, and are generally denoted with a subscript indicating the last peptide residue retained. Thus, EGF lacking the last 2 of its normal 53 peptides is generally indicated as EGF₅₁. Proteins having an amino acid deletion, for example wherein Trp₄₉ is absent, are generally denoted with the term “del” (or .DELTA.) and a subscript indicating the position, without altering the numbering of the remaining amino acids. Thus, if Trp₄₉ were deleted, the resulting protein would be indicated EGF-.DELTA.₄₉. Insertions, increasing the chain length, are generally indicated as substitutions substituting 2 or more amino acids for one, e.g., rhEGF-L/G₁₅ indicates insertion of Gly after the natural Leu₁₅. Finally, an EGF of the invention where His₁₆ has been replaced by another amino acid, with or without other modifications, is generally denoted generically by EGF-X₁₅. Muteins of EGF, as described for example in U.S. Pat. No. 6,191,106 (Mullenbach et al.), which issued Feb. 20, 2001 also fall within this definition provided they have the requisite EGF activity.

“Infection” as used herein means a state or condition in which a pathogenic agent invades the body or a part of it, which under favorable conditions multiplies and produces effects, which are injurious. Pathogenic agents include microorganisms and viruses and infection may be associated with pain, heat, discoloration, swelling and disordered function.

“One milliequivalent (mEq)” as used herein refers to the number of ions in solution as determined by their concentration in a given volume. This measure is expressed as the number of milliequivalents per liter (mEq/l). Milliequivalents may be converted to milligrams by multiplying mEq by the atomic weight of the mineral and then dividing that number by the valence of the mineral.

“Oral rehydration” as used herein means the giving of fluid by mouth to prevent and/or correct the dehydration that is a result of fluid deprivation, excessive loss of fluid, reduction in total quantity of electrolytes, or injection of hypertonic solutions.

“Oral composition” as used herein means any composition taken orally or by nasogastric or oral feeding tube.

“Oral rehydration composition” as used herein means any aqueous oral composition for rehydration, and includes oral rehydration solutions. The oral rehydration composition will comprise a component or components that will actively enhance the absorption of water from the lumen of the gastrointestinal tract. For example, the oral rehydration composition may comprise at least one of the following solutes (i) salts, which dissociate into their respective charged ions/electrolytes in solution for rehydration, (ii) carbohydrates, or (iii) other alternative sodium-coupled nutrient or a source of sodium-coupled nutrients (i.e. amino acids, or a source of amino acids, for example peptides and polypeptides, or short-chain fatty acids or a source of non-digestible carbohydrate which can be metabolized to short-chain fatty acids by bacterial fermentation in the gut). For example, the oral rehydration composition may include Gatorade™, sports drinks and the like.

“Oral rehydration solution” as used herein means an oral rehydration solution (ORS) as is known to those skilled in the art, and includes water, salts which dissociate into their respective charged ions/electrolytes in solution for rehydration, a source of carbohydrates and/or other alternative sodium-coupled nutrient or a source of sodium-coupled nutrients (i.e. amino acids, or a source of amino acids, for example peptides and polypeptides, or short-chain fatty acids or a source of non-digestible carbohydrate which can be metabolized to short-chain fatty acids by bacterial fermentation in the gut), and a source of base.

“Total parenteral nutrition (TPN)” as used herein means providing the total caloric needs by intravenous route for a patient who is unable to take food orally.

The present invention includes a treatment for dehydration. The subject of the treatment may already be suffering from the condition as indicated by any one or more of the following symptoms: watery diarrhea, decreased urinary output, increased urine specific gravity, increased thirst, sunken eyes, loss of skin turgor, dry buccal mucous membranes, depressed anterior fontanel, rapid breathing, lethargy, coma, a rapid weak pulse, hypotension, cold extremities and oligo-anuria.

The present invention includes a treatment for diarrhea. The subject of the treatment may already be suffering from diarrhea or may be at risk of developing diarrhea.

The present invention also includes a method to reduce the severity of diarrhea and promoting intestinal healing. The subject of the treatment may already be suffering from diarrhea or may be at risk of developing diarrhea. The subject may be already suffering from a condition leading to intestinal damage or may be at risk of developing such a condition.

An oral rehydration solution (ORS) comprising EGF may be utilized for any condition in which epidermal growth factor or epidermal growth factor receptor agonists may be beneficial in combination with an ORS. Such conditions may include recovery from gastrointestinal surgery, gastrointestinal resection or small intestinal transplant or other post surgical trauma, short bowel syndrome, burns, oral mucositis, AIDS, inflammatory diseases such as Crohn's disease and Ulcerative colitis, celiac disease, necrotizing enterocolitis, gut prematurity, bone marrow transplants, intestinal damage due to chemotherapy or radiation therapy, sepsis and intestinal infections. Subjects requiring total parenteral nutrition (TPN) may also benefit from receiving the treatment.

Treatment of dehydration and diarrhea involves an epidermal growth factor or an epidermal growth factor receptor agonist. A treatment of the invention may be administered orally, by oral or nasogastric tube, or enterally. The invention can be a composition that may be prepared as a solution, suspension, colloid, concentrate, powder, granules, tablets, pressed tablets, capsules (including coated and uncoated tablets or capsules) and the like. Delayed release or controlled release formulations are also included.

The quantity of epidermal growth factor or epidermal growth factor receptor agonist used in the treatment can vary widely. Typically, the oral composition will comprise from about 20 ng/kg/day (0.0032 nmol/kg/day) to about 20 mg/kg/day (3.2 umol/kg/day), or in the alternative, from about 0.2 ug/kg/day (0.032 nmol/kg/day) to about 2 mg/kg/day (0.32 umol/kg/day), or in the alternative, from about 1 ug/kg/day (0.16 nmol/kg/day) to about 1 mg/kg/day (0.16 mmol/kg/day), or in the alternative, from about 2 ug/kg/day (0.32 nmol/kg/day) to about 0.2 mg/kg/day (32 nmol/kg/day) of epidermal growth factor or epidermal growth factor receptor agonist.

Alternatively, the oral composition will comprise from about 1 ng/kg/day to about 10 mg/kg/day or in the alternative, from about 0.1 ug/kg/day to about 1 mg/kg/day, or in the alternative, from about 1 ug/kg/day to about 1 mg/kg/day, or in the alternative, from about 1 ug/kg/day to about 0.1 mg/kg/day of epidermal growth factor or epidermal growth factor receptor agonist.

Typically, the oral rehydration composition including the ORS, will contain from about 100 picogram epidermal growth factor or epidermal growth factor receptor agonist to about 1 milligram epidermal growth factor or epidermal growth factor receptor agonist per milliliter, or in the alternative, about 1 nanogram epidermal growth factor or epidermal growth factor receptor agonist to about 100 microgram epidermal growth factor or epidermal growth factor receptor agonist per milliliter, or in the alternative, about 10 nanogram epidermal growth factor or epidermal growth factor receptor agonist to about 10 microgram epidermal growth factor or epidermal growth factor receptor agonist per milliliter. It is contemplated that treatment would be administered probably at least once a day, 3 or 4 times per day or more, or even continuously. Intermittent doses could be administered by any convenient route, e.g., bolus infusion or various oral preparations discussed elsewhere herein.

Generally speaking, EGF is prepared by a synthetic process, being manufactured by conventional biotechnological or chemical techniques. EGF might be obtained from a natural source.

The formulations may include additives such as viscosity adjusting agents, osmosity adjusting agents, buffers, pH adjusting agents, flavorings, stabilizers, colorings, preservatives and the like where required.

A treatment of the present invention would require administration by an appropriate route. As the agent is to be administered so as to be biologically available to the free luminal side of epithelial cells of the intestine, oral administration would be most preferred. As such, a unit dose of the agent could be provided in a suitable container for ready opening and delivery to and mixture with a pharmaceutically acceptable solution such as a dietary product or an oral rehydration product. A suitable amount of the components of the present invention could thus be provided in a powdered or granular form to be added directly to a dietary product or liquid suitable for human consumption or an oral rehydration solution. Alternatively, the components of the present invention may be provided as a kit, for example a foil packet comprising EGF. The packet may also comprise salts, carbohydrates, flavourings, etc, in a dehydrated powder form for reconstitution in a given volume of water. The kit may be comprised of an effective amount of EGF, a carbohydrate source such as glucose, fructose or dextrose, an alternative sodium-coupled nutrient or a source of sodium-coupled nutrients (i.e. amino acids, or a source of amino acids, for example peptides and polypeptides, or short-chain fatty acids or a source of non-digestible carbohydrate which can be metabolized to short-chain fatty acids by bacterial fermentation in the gut), a source of sodium, potassium, chloride, a source of base (for example citrate) and instructions. As is known to those skilled in the art, when salts and bases are added to an aqueous solution they disassociate into their respective charged ions/electrolytes. So, while a kit, or the manufacturing process would involve the use of various salts, when constituted into water as a rehydration composition or an ORS, the sodium, chloride, potassium and citrate would be in the form of ions. The kits may be provided for the oral composition, the oral rehydration composition or the oral rehydration solution of the present invention.

Examples of suitable dietary products or liquids include water, saline, buffered solutions, infant formula, and expressed breast milk, other suitable carriers, or combinations thereof. Any solution suitable for oral administration may be used. Additives may be added which act as bystander proteins (i.e. nonactive protein “filler”), which protects the EGF from enzymatic degradation by pancreatic proteases (65). For example, casein (a milk protein) has been used for this experimentally (65). Other approaches may involve administering with a protease inhibitor to preserve EGF structure and activity.

An oral rehydration composition or solution may contain a source of a sodium-coupled nutrient where the absorption of the nutrient is coupled to the active energy driven absorption of sodium. Typically, and in all recommended and commercially produced ORS's this sodium-coupled nutrient is a carbohydrate as the absorption of glucose and galactose (the basic sugar units that along with fructose comprise most carbohydrates and are the basic units into which most complex carbohydrates are digested into prior to absorption) is very tightly bound to the absorption of sodium. Amino acids and sources of amino acids (for example, peptides and polypeptides) may also be used, as well as short-chain fatty acids (these sodium-coupled nutrients are produced endogenously by bacterial fermentation of undigested or non-digestible carbohydrates for example cellulose, in the proximal colon).

Oral rehydration solutions are known to those skilled in the art. The oral rehydration solutions used in this invention may typically contain all the electrolytes at their recommended levels for ORS's sold in the United States or recommended for use in the developing world by the World Health Organization. In addition to sodium (Na⁺), potassium (K⁺), chloride (Cl⁻) and citrate ions, the oral rehydration solutions contain a carbohydrate source such as glucose, fructose or dextrose. The ORS will typically be comprised of water, carbohydrate, sodium ions, potassium ions, chloride ions and citrate ions. Sodium can be added at 20-100 mEq/L with a preferred level of from 40-60 mEq/L for formulations for treatment of acute dehydration and 75-90 mEq/L for formulations for prevention of dehydration or maintenance of hydration. Preferred potassium levels are from 20-30 mEq/L with a broad range of 10-100 mEq/L operable. The chloride anion is normally added at 30-80 mEq/L with a broad range of 25-100 mEq/L operable. The source of base is generally selected from the group consisting of acetate, lactate, citrate or bicarbonate and is normally added at a range of 25-40 mEq/L with a broad range of 10-50 mEq/L operable.

The quantity of sodium ions used in the ORS can vary widely, as is known to those skilled in the art. Typically, the ORS may contain from about 30 mEq/L to about 95 mEq/L of sodium. In a further embodiment, sodium content can vary from about 30 mEq/L to about 70 mEq/, alternatively from about 40 mEq/L to about 60 mEq/L. Suitable sodium sources include but are not limited to sodium chloride, sodium citrate, sodium bicarbonate, sodium carbonate, sodium hydroxide, and mixtures thereof.

The ORS may also contain a source of potassium ions. The quantity of potassium can vary widely. However, as a general guideline, the ORS will typically contain from about 10 mEq/L to about 30 mEq/L of potassium. In a further embodiment, it may contain from about 15 mEq/L to about 25 mEq/L of potassium. Suitable potassium sources include but are not limited to, potassium citrate, potassium chloride, potassium bicarbonate, potassium carbonate, potassium hydroxide, and mixtures thereof.

The ORS may also typically contain a source of chloride ions. The quantity of chloride can vary as is known in the art. Typically the ORS will contain chloride in the amount of from about 30 mEq/L to about 80 mEq/L, or in the alternative, from about 30 mEq/L to about 75 mEq/L, or in the alternative, from about 30 mEq/L to about 70 mEq/L. Suitable chloride sources include but are not limited to, sodium chloride, potassium chloride and mixtures thereof.

The ORS may also contain a source of carbohydrate or a source of amino acids. The quantity of carbohydrate utilized is important. The quantity should normally be maintained at less than about 5% w/w, and in the alternative, about 3% w/w, and in the alternative, about 2.5% w/w. Quantities ranging from about 3% w/w to about 2.0% w/w are suitable. Excessive carbohydrate will exacerbate the fluid and electrolyte losses associated with diarrhea.

Any carbohydrate may be used to practice the present invention. Suitable carbohydrates include, but are not limited to, simple and complex carbohydrates, glucose, dextrose, fructooligosaccharides, fructose and glucose polymers, corn syrup, high fructose corn syrup, sucrose, maltodextrin, and mixtures thereof.

The ORS may also typically include a source of base to replace diarrheal losses. Typically citrate will be incorporated into the ORS to accomplish this result. Citrate is metabolized to an equivalent amount of bicarbonate, the base in the blood that helps maintain acid-base balance. While citrate is the preferred source of base, any base appropriate for or routinely incorporated into rehydration solutions may be used in the practice of the present invention. Citrate has the further benefit of masking the metallic taste of zinc. The quantity of citrate ions can vary as is known in the art. Typically, the citrate content ranges from about 10 mEq/L to about 40 mEq/L, or in the alternative, from about 20 mEq/L to about 40 mEq/L, or in the alternative, from about 25 mEq/L to about 35 mEq/L. Suitable citrate sources include, but are not limited to, potassium citrate, sodium citrate, citric acid and mixtures thereof.

In addition to the sources of carbohydrate detailed above, the ORS may contain rice flour or other components of rice that may be beneficial in the treatment of diarrhea. Rice supplemented oral rehydration solutions are well described in the literature and methods for using such rice supplemented oral rehydration solutions are known to those skilled in the art. Such rice supplemented oral rehydration solutions include those described in U.S. Pat. No. 5,489,440 and U.S. Pat. No. 5,096,894, the contents of which are hereby incorporated by reference.

Rice flour can be made from rice kernels that have been boiled, dehusked, and ground. A rice flour-based oral rehydration solution can be prepared by first adding rice flour to cold or room temperature water while agitating until the mixture contains 6% total solids. To gelatinize the mixture the rice, slurry is heated to 205°-210° F. (96°-99° C.) for 5 to 10 minutes and allowed to cool to 120°-130° F. (49°-55° C.) for enzymatic digestion. To hydrolyze the rice flour, a cellulase and protease is added and hydrolysis allowed to proceed for a period of time that varies depending on the amount of enzyme added. When cellulase is added at 1% by weight of the fiber content of the rice flour and protease is added at 3% by weight of the protein content of the rice flour, one hour is required for hydrolysis. One skilled in the art will be able to determine the enzyme concentrations and hence the time required for hydrolysis under different conditions. The enzymes are inactivated by heating the slurry to 200°-205° F. (94°-96° C.) for 5 to 10 minutes. The slurry is then cooled to 155°-165° F. (68°-74° C.). Carboxymethylcellulose (CMC), minerals and citric acid can be added and the solution homogenized at 4000/500 PSIG (27579.028/3447.3785 kPa).

Alternatively, clarified rice dextrin may be added to the solution to provide from 10 to 80 g/l and typically 10-35 g/L of rice dextrin. A most preferred rice dextrin based ORS comprises per liter: sodium—50 mEq: potassium—25 mEq: chloride-45 mEq: citrate—34 mEq; rice dextrin—30 g. The rice dextrin glucose polymer (GP) profile has a distribution of short-chain glucose polymers consisting of 50 to 90% 2 to 6 glucose units and typically 55 to 80% and in the alternative, 65 to 75% (Wt./Wt. basis).

Rice dextrin suitable for use in the ORS of the invention can be obtained from the solubilized rice starch of Puski et al U.S. Pat. No. 4,830,861 incorporated in entirety herein. Puski et al describe a procedure whereby the carbohydrate in rice flour is solubilized by amylase enzymes and separated from insoluble rice protein and carbohydrate by centrifugation. The resulting soluble fraction contains about 98% carbohydrate and less than 1% but more than 0.1% protein. In order to reduce or eliminate the trace amounts of particulate matter and residual protein which contributes to foaming and browning problems during processing and sterilization and the formation of a fine precipitate during storage, the solubilized rice carbohydrate of Puski et al can be clarified by a process comprising the steps of:

(a) filtering the solubilized rice carbohydrate fraction obtained from rice flour by enzymatic hydrolysis at neutral pH using filter aid at 35° C. to 50° C.: and then
(b) subjecting the filtrate to a second filtration using filter aid and activated carbon at temperatures above 80° C.

If desired, the clarified solution can be spray dried following pH adjustment to 4.0-4.8 and typically 4.5.

Conventional filter aids such as amorphous silica (Silflow™) from Sil Flo Corporation, and activated carbon, Darco S-51™, from American Norit are used. The clarified rice dextrin of the instant process may be spray dried to provide rice dextrin solids having less than 0.1% protein by weight.

Indigestible oligosaccharides may be incorporated into the ORS. Indigestible oligosaccharides have a beneficial effect on the microbial flora of the gastrointestinal tract, promoting the growth of non-pathogenic microbial organisms and inhibiting the growth of pathogenic organisms such as Clostridium difficile. Such ORS's have been described in U.S. Pat. No. 5,733,579, which is incorporated herein by reference. Typically, the oligosaccharide will be a xylooligosaccharide, a fructooligosaccharide, or an inulin such as raftilose. The quantity of indigestible oligosaccharides can vary widely but may range from 1 to 100 grams per liter, and more typically from 3 to 30 grams per liter of aqueous solution.

As used herein “indigestible oligosaccharide” refers to a small carbohydrate moiety (degree of polymerization less than 20 and/or a molecular weight less than 3,600) that is resistant to endogenous digestion in the human upper digestive tract. Indigestible oligosaccharides that may be employed in preferred embodiments of the invention are fructooligosaccharides and xylooligosaccharides. Indigestible oligosaccharides that may be employed in most preferred embodiments of the invention are fructooligosaccharides selected from the group consisting of 1-ketose, nystose and 1^{F}-β-fructofuranosyl nystose fructooligosaccharides, and xylooligosaccharides selected from the group consisting of xylobiose, xylotriose and xylotetrose xylooligosaccharides.

Fructooligosaccharides (FOS) are carbohydrate polymers consisting of a chain of fructose residues linked by (2->1)-β glucosidic bonds and usually carry a single D-glucosyl residue at the non-reducing end of the chain linked (1->2)-α as in sucrose.

FOS occur in nature in many kinds of plants including bananas, tomatoes, onions, wheat, barley, honey, asparagus and artichokes. They can also be synthesized from sucrose through the use of transfructosylating enzymes, such as the enzyme obtained from the fungus Aspergillus niger. Treatment of sucrose with this enzyme results in a mixture of fructooligosaccharides containing 2, 3, or 4 fructose residues. The resulting fructooligosaccharides are designated respectively 1-ketose (GF₂), nystose (GF₃), and 1^{F}-β-fructofuranosyl nystose (GF₄). A method of producing FOS industrially is disclosed in U.S. Pat. No. 4,681,771 to Adachi et al. Xylooligosaccharides (XOS) are prepared by the enzymatic hydrolysis of the xylan from corn, sugar cane, and cottonseed. Xylans are hydrolyzed by a Trichoderma-derived enzyme xylanase to make XOS. Xylooligosaccharides are mainly composed of two, three and four xylose units with a β1-4 linkage, xylobiose, xylobiose, and xylotetrose. Xylobiose, the main component of XOS, is relatively abundant in bamboo shoots.

Zinc may be incorporated into the oral rehydration solution. Zinc reduces the duration and severity of diarrhea and the associated fluid loss. The quantity of zinc used in the ORS of this invention can vary widely. The goal is to provide sufficient zinc to replace the zinc lost due to the underlying diarrhea and/or vomiting. Incorporating from about 0.3 mEq to about 95 mEq of zinc per liter of ORS will typically accomplish this result. Typically, the ORS will contain from about 0.6 mEq to about 3 mEq of zinc per liter. Alternatively, it may contain from about 0.6 mEq to about 1.2 mEq of zinc per liter. The source of zinc ions is not critical. Any zinc salt suitable for human consumption may be used in the ORS of this invention. Examples of suitable zinc sources include zinc gluconate, zinc sulfate, zinc chloride, zinc citrate, zinc bicarbonate, zinc carbonate, zinc hydroxide, zinc lactate, zinc acetate, zinc fluoride, zinc bromide, and zinc sulfonate. Such zinc-supplemented ORS's are described in U.S. Pat. Application 2003/0077333 filed Jun. 4, 2001, the contents of which are hereby incorporated by reference.

Glutamine may also be incorporated into the oral rehydration solution either as N-acetyl-glutamine or a nutritionally acceptable salt thereof. Glutamine has been shown to play a role in intestinal healing and is the primary metabolic fuel for intestinal enterocytes. The term “nutritionally acceptable salt,” means those salts of N-acetyl-L-glutamine which are acceptable for use in a liquid composition that is suitable for administration to humans. Nutritionally acceptable salts of N-acetyl-L-glutamine are salts where the hydrogen of the carboxyl group has been replaced with another positive cation. Such salts can be prepared during the final isolation and purification of the N-acetyl-L-glutamine or separately by reacting the carboxylic group with a suitable base such as the hydroxide, carbonate, or bicarbonate of a metal cation or with ammonia or an organic primary, secondary or tertiary amine. Nutritionally acceptable salt cations may be based on alkali metals or alkaline earth metals such as lithium, sodium, potassium, calcium, magnesium, and aluminum and nontoxic quaternary ammonia and amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine. Such N-acetyl-glutamine supplemented ORS's are described in U.S. Pat. Application 2003/0134851 filed Oct. 8, 2002, the contents of which are hereby incorporated by reference.

An effective amount of N-acetyl-glutamine or a nutritionally acceptable salt thereof is typically an amount sufficient to provide approximately 10-50 g of total glutamine per day or alternatively at least about 140 mg total glutamine per kg of body weight per day, or in the alternative, at least 250 mg total glutamine per kg of body weight per day (mg/kg/day). The N-acetyl-L-glutamine will provide from about 1-100% of the total glutamine that the patient consumes on a daily basis, typically from about 10-95%, or in the alternative, from about 75-90% of the total glutamine that the patient consumes on a daily basis.

When N-acetyl-L-glutamine or a nutritionally acceptable salt thereof provides the sole source of glutamine that the patient consumes, an effective amount of N-acetyl-L-glutamine or a nutritionally acceptable salt thereof is typically at least about 0.7 mmoles/kg/day. In the alternative, an effective amount of N-acetyl-L-glutamine or a nutritionally acceptable salt thereof may be at least about 1.0 mmoles/kg/day. An effective amount of N-acetyl-L-glutamine may be at least about 1.5 mmoles/kg/day.

As noted above, the amount of N-acetyl-L-glutamine or a nutritionally acceptable salt thereof needed to provide total glutamine of 250 mg/kg/day will vary depending upon the amount of glutamine present in any other protein components the patient is consuming. As a general guideline, the patient should consume at least about 0.7 to about 4.0 mmoles of N-acetyl-L-glutamine or a nutritionally acceptable salt thereof per kg per day to obtain the full benefits of this invention. Lesser amounts may be beneficial, depending on the total glutamine content of the other components of the protein system. In general, sufficient N-acetyl-L-glutamine should be provided to the patient to deliver at least about 140 mg of total glutamine per kg of body weight per day, typically at least about 250 mg total glutamine per kg of body weight per day.

The quantity of N-acetyl-L-glutamine that may be incorporated into an aqueous solution, such as ORS, can vary widely. Typically, the ORS will contain at least about 5.0 mmoles of N-acetyl-L-glutamine or a nutritionally acceptable salt thereof per liter of solution, and further contain at a minimum, water, glucose, and sodium. Typically, the ORS will contain about 20 to about 300 mmoles per liter of N-acetyl-L-glutamine or a nutritionally acceptable salt thereof, and more typically from about 25 to about 200 mmoles. If a liquid such as Kool-Aid™ or Gator-Aid™ is utilized, then the quantity of N-acetyl-L-glutamine will be comparable to that described for the ORS.

Alternatively, stable glutamine derivatives can be prepared by coupling glutamine with one or more additional amino acids to provide oligopeptides, or with glucose, or both, or acylating glutamine with a carboxylic acid having 2 to 6 carbon atoms, to provide a compound which is stable to degradation under acidic environments. While any naturally occurring amino acid may be used as the additional amino acid coupled to the glutamine, it is preferred to use alanine or glutamine, alone or in combination as the additional amino acids. A preferred number of total amino acid groups present in the compounds used in the present method ranges from 2 to 5 (formed from coupling from 1 to 4 amino acids with glutamine), with dipeptides and tripeptides most preferred. Most preferred compounds include alanyl-glutamine, alanyl-glutaminyl glutamine and gamma-glutamyl glutamine. The compounds are known and can be prepared using conventional peptide coupling reactions, such as on a solid phase peptide synthesizer or using 1,3-diisopropyl-carbodiimide (DIPCDI) activation in solution coupling.

Stable glutamine derivatives are administered at a dose range effective to bring about improved intestinal sodium cotransport. A preferred dosage range of glutamine equivalent (Gln has a molecular weight of 146) is 0.05 to 0.8 g/kg/day of patient body weight, with approximately 0.5 to 0.6 g/kg/day or solutions of approximately 13 g/L glutamine equivalent (the solutions have sufficient glutamine derivative to provide an effective glutamine level equivalent to a solution of 13 g/L glutamine) or 1-10 mM glutamine derivatives being most preferred. Stable glutamine derivatives are described in U.S. Pat. No. 5,561,111 the contents of which are hereby incorporated by reference.

U.S. Pat. Nos. 4,505,926, 4,539,319, 4,558,063 and 4,594,195 disclose various oral rehydration solutions containing pharmaceutically active ingredients (i.e., drugs) for treatment of enterotoxin induced diarrhea and prevention of death from enteropathogenic E. coli infection of the gastro-intestinal tract. Drugs incorporated into these prior art rehydration solutions include quaternary aminophenyliminoimidazolidines, 2-aminoimidazoline derivatives, and 5,6,7,8-tetrahydronaphonitrile intermediates.

Quaternary aminophenyliminoimidazolidines are useful in treating diarrhea in humans and scours in animals, particularly to treatment of enterotoxin induced diarrhea. The amount of drug administered must, of course, be sufficient to bring about the desired effect and will also depend on the body weight of the recipient and the chosen route of administration. Typical dosages are in the range from 0.1 to 100 mg/kg particularly from 1 to 50 mg/kg. Useful dosage units based on such dosage would contain from 0.1 mg to 2500 mg of the drug, more suitably 1 mg to 2500 mg. Of course, it will be appreciated that many preferred compositions of the invention are in multi-dose form as, for the therapy of animals, it is often most desirable to be able rapidly to treat a number of animals. Such multi-dose compositions will contain, by way of example, at least 10 mg of the drug. Depending on the exact nature of the said multi-dose composition, often it will contain at least 250 mg of the drug, and on occasions as much as 25 g. Doses may be administered once or several times daily. Quaternary aminophenyliminoimidazolidines are described in U.S. Pat. No. 4,505,926 which is hereby incorporated by reference.

A number of novel amidine derivatives have been shown to inhibit enterotoxin-induced secretion into the small intestine and are, therefore, useful in treating enterotoxin induced diarrhea in humans and scours in animals. The amount of drug administered must, of course, be sufficient to bring about the desired effect and will also depend on the body weight of the recipient and the chosen route of administration. Typical dosages are in the range from 0.1 to 100 mg/kg particularly from 1 to 10 mg/kg. Useful dosage units based on such dosage would contain from 0.1 mg to 5 g of the drug, more suitably 1 mg to 500 mg. Of course, it will be appreciated that many preferred compositions of the invention are in multi-dose form as, for the therapy of animals, it is often most desirable to be able rapidly to treat a number of animals. Such multi-dose compositions will contain, by way of example, at least 1 mg of the drug. Depending on the exact nature of the said multi-dose composition, often it will contain at least 50 mg of the drug, and on occasions as much as 50 g. Doses may be administered once or several times daily. Novel amidine derivatives are described in U.S. Pat. No. 4,539,319 which is hereby incorporated by reference.

2-aminoimidazoline derivatives inhibit small-intestinal secretion whilst having less CNS activity than alpha-agonists of similar structure. The compositions of the invention will contain sufficient drug to enable this effective dose to be administered in a convenient manner. Thus by way of example useful dosage units of the composition may contain 1 μg to 50 mg of the drug, more suitably 20 μg to 20 mg. Of course, it will be appreciated that many preferred compositions of the invention are in multi-dose form, as for the therapy of animals, it is often most desirable to be able rapidly to treat a number of animals. Such multi-dose compositions will contain by way of example, at least 1 mg of the drug. Depending on the exact nature of the said multi-dose composition, often it will contain at least 50 mg of the drug, and on occasions as much as 1 g. 2-aminoimidazoline derivatives are described in U.S. Pat. No. 4,558,063 which is hereby incorporated by reference.

5,6,7,8-tetrahydro-napthonitrile intermediates inhibit enterotoxin-induced secretion into the small intestine and are, therefore, useful in treating enterotoxin induced diarrhea in humans and scours in animals. The amount of drug administered must, of course, be sufficient to bring about the desired effect and will also depend on the body weight of the recipient and the chosen route of administration. Typical dosages are in the range from 0.1 to 100 mg/kg particularly from 1 to 10 mg/kg. Useful dosage units based on such dosage would contain from 0.1 mg to 5 g of the drug, more suitably 1 mg to 500 mg. It will be appreciated that many preferred compositions of the invention are in multi-dose form as, for the therapy of animals, it is often most desirable to be able rapidly to treat a number of animals. Such multi-dose compositions will contain, by way of example, at least 1 mg of the drug. Depending on the exact nature of the said multi-dose composition, often it will contain at least 50 mg of the drug, and on occasions as much as 50 g. Doses may be administered once or several times daily. 5,6,7,8-tetrahydro-napthonitrile intermediates are described in U.S. Pat. No. 4,594,195 which is hereby incorporated by reference.

U.S. Pat. No. 4,942,042 is directed to an anti-diarrhea composition comprising an absorptive component and an electrolyte/sugar component. The absorptive material is a thermally activated, finely powdered, hydrous magnesium aluminum silicate clay capable of absorbing pathogenic intestinal bacteria. The absorptive material is also capable of absorbing diarrhea-associated viruses, intestinal toxins and gases. Suitable absorptive materials are clays such as Smectite (Si₈Al₄O₂₀OH₄). Other such clays are argillaceous clays, for example the clay known in its unactivated form as mormoiron attapulgite further named ATTA under its activated form. This is also well-known as an anti-diarrhea absorptive material.

The absorptive material is provided in an amount recognized as effective in the treatment of diarrhea, in a package whose contents are to be reconstituted with 200 ml of water. The absorptive material is provided in an amount such that, following reconstitution, it is present in a concentration of 2.5-15 g/l. The composition is packaged in solid form and reconstituted by admixture with water prior to administration. U.S. Pat. No. 4,942,042 is hereby incorporated by reference.

U.S. Pat. No. 5,192,551 discloses rehydration and infant nutrient formulas containing a neutral glycolipid, in particular, gangliotetracosylceramide. The glycolipid binds enteric virus, e.g., rotaviruses, which are pathogenic to humans. Rotaviruses are RNA viruses known to replicate in the intestinal epithelial cells of a wide range of animal species, including humans. The GA1 glycolipid may be obtained commercially, for example from the Sigma Chemical Co., St. Louis, Mo. GA1 can also be prepared by acid hydrolysis of GM1 glycolipid as described in Dahms, et al., (28). As this neutral glycolipid is acid-stable, it does not need to be protected to survive in the gastrointestinal tract. However, it may be desirable to bind the neutral glycolipid to a non-absorbable support. The selection of a support is within the skill of the art and such supports include beads, resins, natural or synthetic polymers. One particularly preferred non-absorbable support is cholestyramine, which has previously been shown to be effective as an antidiarrheal agent for bacterial pathogens. The neutral glycolipid may be bound to the non-absorbable support via a simple absorption or via a covalent linkage. Methods for performing both such types of binding are well known in the art. (See, e.g., Tiemeyer, et al., (81) and Taki, et al., (80)) If absorption is to be used as the means of attachment, a hydrophobic bead is preferred. U.S. Pat. No. 5,192,551 is hereby incorporated by reference.

An effective amount of ganglidtetraosylceramide according to the present invention is one that is effective to bind to the targeted viruses. Generally, for oral administration, this will be between about 10 μM and about 1 mM. Between about 12 μg and 1.2 mg will be administered to a child and between about 200 μg and 10 mg will be administered to an adult.

The ORS of this invention can be manufactured using techniques well known to those skilled in the art. As a general guideline, all the ingredients may be dry blended together; dispersed in water with agitation; and optionally heated to the appropriate temperature to dissolve all the constituents. The ORS is then packaged and sterilized to food grade standards as is known in the art.

Optionally, preservatives may be added to extend the shelf life. The appropriate preservative and the concentration needed to accomplish this result will be known to those skilled in the art. Typical preservatives can include, but are not limited to, potassium sorbate and sodium benzoate.

The ORS of the present invention will also typically include a flavor to enhance its palatability, especially in a pediatric population. The flavor should mask the salty notes of the ORS. Useful flavorings include, but are not limited to, cherry, orange, grape, fruit punch, bubble gum, apple, raspberry and strawberry. Artificial sweeteners may be added to complement the flavor and mask the salty taste. Useful artificial sweeteners include saccharin, Nutrasweet™, sucralose, acesulfane-K (ace-K), etc.

Although the above mentioned components have been described with reference to an ORS, it is to be understood that the oral composition and oral rehydration composition of the present invention can also comprise these components. For example, the oral rehydration composition may comprise EGF, a source of carbohydrates, sodium, potassium and zinc. Alternatively, the oral rehydration composition may comprise EGF, amino acids and sodium. The oral composition may comprise an EGF receptor agonist, sodium and an absorptive component.

Depending on patient preference, the oral composition, oral rehydration composition or ORS may be administered in various forms known to those knowledgeable in the art. Some children will consume oral compositions, oral rehydration compositions and ORSs more readily, for example, if frozen, such as in the form of a Popsicle. Following preparation of the oral composition, the product is encapsulated within a sealable freezable packaging material and sealed such as by heat sealing. In a preferred embodiment of the invention, a single dose of composition is packaged in a hermetically sealed freezable pouch. Various types of packaging materials which can be used to practice the invention, such as that used in traditional freezer pops, would be readily apparent to the skilled artisan. The wrapping material is typically a type which will allow markings, such as product identification, ingredients, etc., to be placed on the exterior surface thereof. The formulation is shipped and stored, in multiple units thereof, in this condition. It is contemplated that multiple units or freezer pops will be packaged together for purposes of commercialization.

Prior to administration, for example, a package of liquid oral composition, oral rehydration composition or oral rehydration solution is frozen. Following freezing, the package is opened and the contents thereof eaten. Since the frozen composition/solution will normally be administered at ambient temperatures, the amount of liquid contained in each package is typically an amount which can be consumed in its entirely while still in the frozen state. Typically 20-35 ounces, or in the alternative, 2.0 to 2.5 ounces per package. In a particularly preferred embodiment, 2.1 ounces of sterile composition is encapsulated within an rectangular, e.g., 1″×8,″ freezable wrapper material. Clear plastic wrapper material is preferred.

The frozen formulation of the invention is eaten in the same way as a traditional freezer pop. While the invention will hereinafter be described in terms of a rehydrating freezer pop, it is to be understood that other packaging systems for the delivery of frozen compositions are also encompassed by the invention and are within the scope thereof. Oral rehydration solution Popsicles are described in U.S. Pat. No. 5,869,459, the contents of which are hereby incorporated by reference.

Oral compositions can also be formed into gels to enhance patient compliance. Gelled oral rehydration solutions are described in U.S. Pat. No. 6,572,898 the contents of which are hereby incorporated by reference. The oral composition, aqueous oral rehydration composition and ORS may be formed into a flowable gel or alternatively formed into a self-supporting gel structure. Suitable gelling agents include but are not limited to agar, alginic acid and salts, gum arabic, gum acacia, gum talha, cellulose derivatives, curdlan, fermentation gums, furcellaran, gelatin, gellan gum, gum ghatti, guar gum, iota carrageenan, irish moss, kappa carrageenan, konjac flour, gum karaya, lambda carrageenan, larch gum/arabinogalactan, locust bean gum, pectin, tamarind seed gum, tars gum, gum tragacanth, native and modified starch, xanthan gum and mixtures thereof. Usage rates of said gelling agents range from about 0.05 to about 50 wt./wt. %. The appropriate gelling agent and the concentration needed to accomplish this result will be known to those skilled in the art.

For example, the gel oral composition of the invention may be made by first preparing a an oral composition, an oral rehydrating composition or an oral rehydrating solution. A structuring agent is then added to the composition/solution. As used herein, the term structuring agent means any gelling or thickening agent that can be combined with other necessary ingredients to provide a gel composition having a preferred product consistency at room temperature. As used herein, the term preferred product consistency at room temperature is defined as having a gel strength in the range of from about 20 to about 1000 grams per cm², and typically from about 100 to about 200 grams per cm². Gel strength is measured by any convenient method known to those in the art, and typically is measured by a gelometer.

In preparing a gel composition for a typical gel strength test, a portion of water is heated and all non-water ingredients are added. The mixture is stirred at elevated temperature until all non-water ingredients, including the structuring agent, are dissolved. Finally, the remaining water is added and the resulting mixture stirred with continued heating to about 185° F. The mixture is then removed from heat and poured into four 50 ml beakers, each filled to the 40 ml mark. The beakers are placed in a refrigerator at 45° F. and equilibrated for two hours. The beakers are then removed from the refrigerator and gel strength readings are immediately taken using a Stevens 1CM2 gelometer. The measured gel strength is the average of the four readings.

Exemplary structuring agents include but are not limited to agar, alginates, carrageenan, in kappa, iota, or lambda form, cellulose derivatives, exudate gums, gellan gum, gelatin, guar gum, konjac gum, locust bean gum, microcrystalline cellulose, modified starches, pectins, seed gum, and xanthan gum. Preferred structuring agents include but are not limited to gelling agents such as agar, alginate, carrageenan, and pectin. Preferred structuring agents also include but are not limited to thickening agents such as gum arabic, gum tragacanth, tamarind gum, taragum, guar, locust bean gum, and xanthan gum. A most preferred structuring agent is Ticagel™ 550, available commercially from TIC Gums Inc., Belcamp, Md. Ticagel™ 550 comprises a blend of carrageenan and locust bean gum and produces a preferred composition consistency at room temperature when used in an amount of about 4.5 grams per liter.

Any numerical range should be considered to provide support for a claim directed to a subset of that range. For example, a disclosure of a range of from 1 to 10 should be considered to provide support in the specification and claims to any subset in that range (i.e. ranges of 2-9, 3-6, 4-5, 2.2-3.6, 2.1-9.9, etc.). Any reference in the specification or claims to a quantity of an electrolyte should be construed as referring to the final concentration of the electrolyte in the ORS. Tap water often contains residual sodium, chlorine, etc. A value of 40 mEq of sodium, in this application, means that the total sodium present in the ORS equals 40 mEq, taking into account both added sodium as well as the sodium present in the water used to manufacture the ORS. This holds true for all electrolytes and components of the treatment.

A treatment of an animal with an ORS comprising EGF could follow conventional rehydration therapy regimens. Such animals would include patients suffering from diarrhea, patients kept too long on intravenous fluids or tube feeding, or those having damaged or functionally impaired intestinal mucosa from infection, chemotherapy or surgical intervention. The need for initiating treatment in rehydration therapy is judged by conventional standards. The therapy is continued until the patient has improved beyond the minimum hydration requirements or as long as indicated by clinical assessment. Animals that may benefit from a treatment of the invention include but are not limited to humans, farm animals such as horses, cows, pigs, goats, sheep, rabbits, mink, llamas, alpacas, elk, bison, fish and poultry, and domestic animals such as cats and dogs. The quantity of epidermal growth factor or epidermal growth factor receptor agonist used in the treatment can vary widely. The animals may be treated with an oral composition, an oral rehydration composition or an ORS supplemented with EGF, an EGF receptor agonist or pharmaceutically acceptable salt forms thereof of the present invention. For example, typically, the ORS will contain from about 100 picogram epidermal growth factor or epidermal growth factor receptor agonist to about 1 milligram epidermal growth factor or epidermal growth factor receptor agonist per milliliter, or in the alternative, about 1 nanogram epidermal growth factor or epidermal growth factor receptor agonist to about 100 microgram epidermal growth factor or epidermal growth factor receptor agonist per milliliter, or in the alternative, about 10 nanogram epidermal growth factor or epidermal growth factor receptor agonist to about 10 microgram epidermal growth factor or epidermal growth factor receptor agonist per milliliter. It is contemplated that treatment would be administered probably at least once a day, 3 or 4 times per day or more, or even continuously. Intermittent doses could be administered by any convenient route, e.g., bolus infusion or various oral preparations discussed elsewhere herein. Epidermal growth factor receptor agonists may include but are not limited to EGF, transforming growth factor alpha, amphiregulin, heparin binding EGF, and epiregulin.

Experiments performed, which establish the feasibility of this invention as an effective treatment are described below.

### Example 1

### Use of EGF to Treat Infectious Malabsorptive Diarrhea

These studies utilized an established model of enteropathogenic E. coli infection in rabbits (20). Oral rehydration therapy is dependent upon the coupled absorption of sodium and glucose in the small intestine. During malabsorptive diarrheal disease glucose transport is impaired. While previous studies have demonstrated that epidermal growth factor (EGF) when applied to the epithelial surface of the jejunum, rapidly increases glucose, sodium and water absorption from the lumen (38;61), it was not known whether EGF could increase intestinal absorption in the presence of an ongoing diarrheal disease which produces disturbances in transport function.

### Methods

Rabbits infected with attaching-effacing E. coli (RDEC-1) were used (20). Briefly, experimental rabbits (400-600 g) were orally inoculated with 5×10⁷ live E. coli in 1 mL 10% sodium bicarbonate. Controls received sodium bicarbonate only. Animals were housed individually in a level B containment room, fed commercial feed and given water ad libitum. Ten days post-infection, all animals were killed with an intra-peritoneal overdose of sodium pentabarbitone following anesthesia with halothane. A 20-cm segment of jejunum beginning 5 cm distal to the ligament of Treitz was then removed and rinsed with ice-cold Kreb's buffer. Both longitudinal and circular muscle layers were stripped from the underlying mucosa and four adjacent pieces of jejunal tissue mounted under short-circuited conditions in Ussing chambers. Both mucosal and serosal surfaces were bathed in 10 ml of oxygenated Kreb's buffer with 10 mM glucose added to the serosal surface to provide metabolic energy to the tissue, 10 mM mannitol added to the mucosal surface to osmotically balance the glucose solution, and 20 mM 3-O-methyl glucose added to both surfaces. Immediately after mounting, 10 μCi 3-O-methyl[³H]glucose was added to either the serosal or mucosal surface and allowed to equilibrate for 20 min. 3-O-methyl glucose is a non-metabolized glucose analog. Unidirectional mucosal to serosal (Jₘₛ) and serosal to mucosal (Jₛₘ) fluxes were then determined by measuring 3 consecutive 5 min flux intervals and an overall 15 min flux interval before and after the addition of 100 ng/ml human recombinant epidermal growth factor (rhEGF) (Austral Biologicals, San Ramon, Calif.) or vehicle to the mucosal reservoir. Net flux (Jₙₑₜ) was calculated by subtracting the serosal to mucosal flux, Jₛₘ, from the mucosal to serosal flux, Jₘₛ. Clinical symptoms were monitored including fecal shedding of the bacterium.

### Results

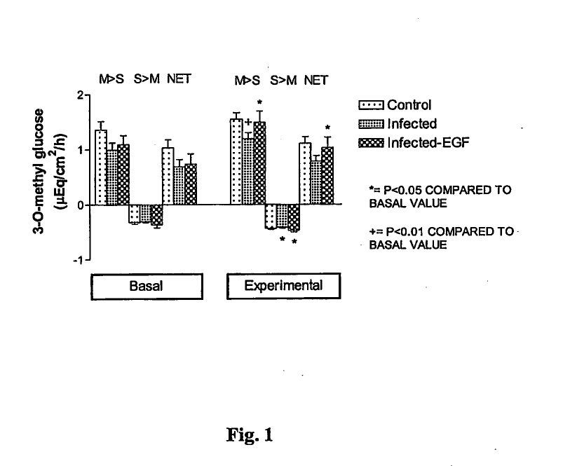

All infected animals used in this study were positive for fecal excretion of RDEC-1 on the day of sacrifice. No control animals tested positive for RDEC-1. As shown in FIG. 1, infection resulted in a significant decrease in net 3-O-methyl glucose transport due to a decrease in the mucosal to serosal flux of 3-O-methyl glucose. Application of mucosal EGF significantly increased the mucosal to serosal and net flux of 3-O-methyl glucose in infected tissue. Mucosal to serosal and net 3-O-methyl glucose transport did not differ in infected tissue-treated with EGF from flux values obtained from controls.

### Conclusions

The work performed in a rabbit model of malabsorptive disease demonstrated that, when applied to the lumenal surface of the intestine, EGF rapidly reversed the impairment of glucose absorption observed in untreated, sick animals. As intestinal water absorption and clinical rehydration is closely coupled to the absorption of nutrients from the intestine, these studies established the proof of concept for the use of EGF in an enhanced oral rehydration solution.

### Example 2

### Use of EGF in an Oral Composition to Reduce the Duration of Diarrhea

Experiments were performed examining the effect of EGF administered in an oral composition on the duration of diarrhea in a model of enteropathogenic E. coli infection in piglets.

### Methods

An enteropathogenic Escherichia coli isolate obtained from Veterinary Pathology Laboratories (VPL), Edmonton, Alberta was utilized in these experiments. This strain was originally isolated from a piglet with diarrhea and is positive for Stb, Lt, and K88 (F4). This strain is referred to as K88 positive E. coli. Enteropathogenic E. coli infections produce primarily a malabsorptive diarrhea, although there is a smaller secretory component.

Bacteria were grown in LB™ broth (Difco Laboratories, Detroit, Mich.) and frozen on Microbank™ porous beads (Pro-Labs Diagnostics, Richmond Hill, ON) at −70° C. For each inoculum preparation, one bead was thawed and incubated in 20 ml sterile LB™ broth for 5 h at 37° C., 200 rpm. Then, the 20 ml starter culture was placed in 980 ml LB™ broth and incubated at 37° C. for 15 hours, 200 rpm. LB™ broth was aliquoted in 18 sterile polystyrene 50 ml tubes (Falcon), centrifuged at 3000 rpm (floor Sorval) for 10 minutes, and all pellets pooled and resuspended in 50 ml 10% NaOHCO₃-sterile PBS.

Inoculum: Piglets received late log phase 1×10¹⁰ (counted by spectrophotometry, 600 nm, A=1.9) live K88 positive E. coli. Bacterial inoculum was delivered in 10% NaHCO₃/sterile PBS (5 ml), followed by lactate Ringer's solution (5 ml). The inoculum (10 ml) was delivered via a 40 cm orogastric intubation tube connected to a 20 cc syringe. Total bacterial counts in inoculum were calculated via serial dilutions plated onto LB™ agar and incubated overnight at 37° C., 5% CO₂.

A pregnant sow was purchased from a local commercial supplier (Doug Hall, Airdrie, AB) approximately one week prior to due-date. The sow was housed at the University of Calgary farm in a farrowing pen. The sow was given free access to water and pig feed (Doug Hall, Airdrie, AB). Two-day old piglets were weight ranked and randomly assigned to each experimental group (see below), and treatment was initiated that day (Day-1). Inoculation with live K88 positive E. coli was carried out the following day (Day 0). Piglets with their sow were housed separately in contained animal facilities for the duration of the experiment at the University of Calgary farm. Housing was at 20° C., with approximate 8:16 h photoperiods.

Piglets were assigned to one of the following experimental groups: 1) infected-untreated, inoculated with 1×10¹⁰ live K88 positive E. coli in 5 mL 10% NaHCO₃— sterile PBS, followed with 5 mL Lactated Ringers at 3 days of age (Day 0). One day prior to infection and daily thereafter, piglets were gavaged orally with sterile PBS, and 2) infected EGF-treated, inoculated with 1×10¹⁰ live K88 positive E. coli in 5 mL 10% NaHCO₃-sterile PBS, followed with 5 mL Lactated Ringers at 3 days of age (Day 0). One day prior to infection and daily thereafter, piglets were gavaged orally with rhEGF in sterile PBS at a concentration of 100 μg/kg body weight.

The study was terminated on Day 10. A total of 11 piglets were studied, 5 were given EGF daily, and 6 received vehicle (untreated).

Piglets were checked daily for weight gain, production of diarrhea, and overall clinical condition (alertness, body posture, condition). In addition, daily fecal scoring was carried out twice daily (9:00 am, and 3:00 pm) as follows: 0=solid, formed stools, 1=soft stools with no perineal soiling, 2=non-bloody diarrhea with perineal soiling, 3=bloody diarrhea with perineal soiling.

All morning observations were performed between 7:00 am and 12:00 pm to avoid diurnal variations. One additional fecal scoring was carried out at 3:00 pm.

### Results

Each piglet received 1×10¹⁰ live K88 positive E. coli, as demonstrated by serial dilutions of the inoculum grown on LB™ agar plates overnight. Absorbance for this inoculum was 1.9 (read at 600 nm).

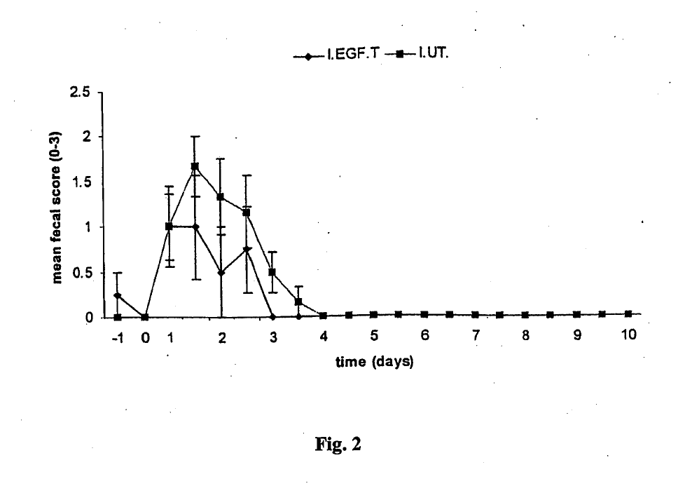

Inoculation with 1×10¹⁰ live K88 positive E. coli induced disease in neonatal piglets. Infected animals became lethargic within 24 h of infection and suffered from diarrhea, which was confirmed by increased fecal scores (FIG. 2). No animal became soiled with bloody diarrhea, and the highest fecal score measured throughout this study was 2. Fecal scores returned to normal in EGF-treated animals (i.e. a fecal score of 0 in all animals) by Day 3, while fecal scores returned to normal in untreated animals (i.e. a fecal score of 0 in all animals) by Day 4 (FIG. 2). Weight gain did not differ between any of the groups over the course of the study.

In summary, experimental inoculation with live K88 positive E. coli causes diarrhea in healthy neonatal piglets. EGF treatment accelerated the return to normal fecal consistency in all treated animals by one day (by day 3 instead of by day 4 in untreated animals).

### Conclusions

In neonatal piglets suffering from diarrhea, therapeutic daily oral administration with EGF accelerates recovery and elimination of diarrheal symptoms and lessens the duration of diarrhea.

### Example 3

### Use of EGF in an Oral Composition to Treat and Reduce the Severity of Diarrhea

These studies examined the effect of treatment with EGF in an oral composition on the severity and duration of established diarrhea.

### Methods

A pregnant sow was purchased from a local commercial supplier (Doug Hall, Airdrie, AB) approximately one week prior to due-date. The sow was housed at the University of Calgary farm in a farrowing pen. The sow was given free access to water and pig feed (Doug Hall, Airdrie, AB). Two-day old piglets were weight ranked and randomly assigned to each experimental group (see below), and treatment was initiated that day (Day 0) and animals assessed 24 hr later. Piglets demonstrated signs of spontaneous neonatal diarrhea at time of enrollment. Piglets with their sow were housed separately in contained animal facilities for the duration of the experiment at the University of Calgary farm. Housing was at 20° C., with approximate 8:16 h photoperiods.

Piglets were assigned to one of the following experimental groups: 1) untreated animals gavaged orally with 5 mL sterile PBS daily starting on Day 0, and 2) EGF-treated, gavaged orally with rhEGF in 5 mL sterile PBS at a concentration of 100 μg/kg body weight daily starting on Day 0.

The study was terminated on Day 1. A total of 10 piglets were studied, 5 were given EGF, and 5 received vehicle (untreated).

Piglets were checked daily for weight gain, production of diarrhea, and overall clinical condition (alertness, body posture, condition). In addition, daily fecal scoring was carried out twice daily (9:00 am, and 3:00 pm) as follows: 0=solid, formed stools, 1=soft stools with no perineal soiling, 2=non-bloody diarrhea with perineal soiling, 3=bloody diarrhea with perineal soiling.

All morning observations were performed between 7:00 am and 12:00 pm to avoid diurnal variations. One additional fecal scoring was carried out at 3:00 pm.

### Results

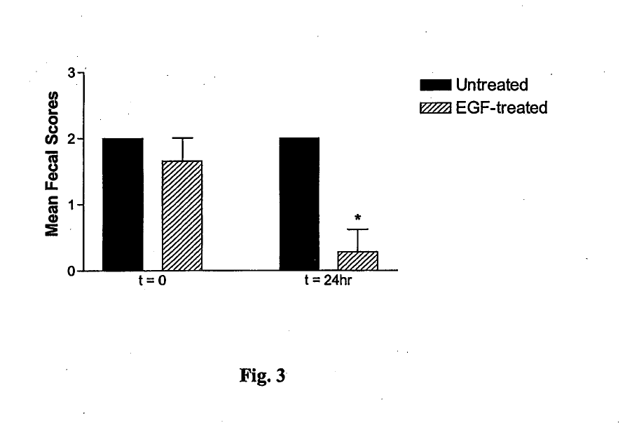

Nine out of 10 piglets had neonatal diarrhea (fecal score of 2 in 7 animals, and score of 1 in 1 animal) of unknown etiology by 2 days of age (Day 0). The following day (Day 1), 3 animals had died. Of these, 1 piglet had received EGF the previous day, while 2 were untreated. On the day of group allocation and treatment initiation, fecal scores of animals in both groups were high (neonatal diarrhea) but not different between both groups (FIG. 3). One day after initiation of treatment (Day 1), piglets given EGF had significantly (P<0.05) reduced their fecal scores compared with piglets given vehicle only (FIG. 3). Weight gain did not differ between the two study groups. In summary, in animals presenting with neonatal diarrhea of unknown etiology, therapeutic administration of EGF significantly reduced the severity of diarrhea within 24 hours.

### Conclusions

In neonatal piglets presenting with neonatal diarrhea of unknown etiology, therapeutic administration of EGF to treat an ongoing infection significantly reduced the severity of diarrhea within 24 hours.

### Example 4

### Use of ORS Supplemented with EGF to Enhance Rehydration, Treat Diarrhea and Promote Intestinal Healing

These studies utilized an established model of enteropathogenic E. coli infection in rabbits (20). Experiments examined physiological parameters of rehydration and assessed the effect of EGF supplemented ORS on diarrhea and intestinal healing in infected animals.

### Methods

Escherichia coli: Enteropathogenic E. coli ATCC 49106 (RDEC-1) was obtained from the ATCC. Freeze-dried bacteria were re-hydrated into 4 mL of Tryptic Soy Broth (TSB) with 10% Fetal Bovine Serum (FBS), following ATCC standard procedure. After overnight growth at 37° C., the broth culture was streaked for single colonies onto MacConkey agar (Difco Laboratories, Detroit, Mich.) plates and incubated overnight at 37° C. Approximately 10 colonies were used to inoculate 125 mL of TSB with 10% FBS and incubated for 19 hours in a 37° C. shaking incubator. The broth was diluted until the ocular density (OD₄₀₅) was 1.45 (by adding 125 mL sterile TSB with 10% FBS). The final inoculum was prepared by adding 4 mL of the diluted culture into 80 mL of 10% NaHCO₃ in PBS. The resulting number of bacteria given to each animal was in the order of 1.232×10⁸ given in 2.5 mL of 10% NaHCO₃.

Bacteria were prepared for long-term storage by adding 20% v/v glycerol to an overnight broth culture prepared as for the above inoculum with no dilution, and freezing/storing at −70° C. in 500 uL aliquots.

A growth curve of the bacterial isolate was obtained prior to infecting rabbits. Live bacterial numbers were calculated from serial dilutions on MacConkey agar at various time points between 0 and 24 hours post inoculation in TSB with 10% FBS prepared as previously reported (20). Briefly, bacterial broth cultures were initiated as described above for preparing the inoculum. At 6, 19, and 24 hours the broth cultures were serially diluted in sterile PBS and spot plated with 20 uL onto MacConkey agar plates. Colonies were counted after overnight incubation at 37° C. and Colony Forming Units (CFU) were calculated for each time point. In addition, the ocular density (OD₄₀₅) of the broths were measured at each time point.

Experimental design: Weanling 4-week-old New Zealand White rabbits (Vandermeer Inc., Edmonton, AB) were obtained and acclimated in the Life and Environmental Sciences Animal Resources Centre (LESARC) facilities at the University of Calgary (Calgary, AB, Canada) for 4 days prior to study. At the initiation of the study all animals appeared healthy and no animals had any evidence of diarrhea. Rabbits were infected with a noninvasive, attaching effacing E. coli 015 (RDEC-1) obtained from the American Type Culture Collection as previously reported with the exception that the animals were inoculated with a higher dose (˜10⁸) of live E. coli RDEC (20) as described above. Animals were assigned to one of three groups:
1) uninfected sham-treated controls orally gavaged with 5 mls vehicle (sterile PBS) only;
2) infected, orally gavaged with 5 mls of standard ORS;
3) infected, orally gavaged with 5 mls of standard ORS supplemented with 12 μg/ml of recombinant human EGF (rhEGF) for a total of 60 μg EGF in 5 mls.

Animals were infected on day 0 and were orally gavaged once a day with the various treatments from day 3 to day 7 post-infection (PI). Uninfected controls were sham gavaged. On day 7 PI animals were killed by lethal intra-cardiac injection with ©Euthanyl (Pentobarbital Sodium) and blood and small intestinal tissue obtained for further analysis. Seven control animals were run and six animals were run per infected group. Recombinant human EGF obtained from Protein Express (Japan) was utilized for these studies. Animals were given water and feed (chow) ad libitum. The ORS formulation utilized was the World Health Organization's recommended low osmolality oral rehydration solution (88). Briefly, ORS was prepared as follows: 2.6 g NaCl, 13.5 g Glucose, 1.5 g of KCl, and 2.9 g of Na₃C₆H₅O₇ was dissolved into 1 L of double distilled water.

Measurements: Weight gain, food intake, and fecal scoring were recorded daily. Fecal scoring was done on a 3 point scale: 1) normal; 2) light diarrhea/soft pellets; 3) heavy diarrhea with perineal soiling. Fecal passage of E. coli RDEC-1 was assessed from daily rectal swabs plated onto MacConkey agar. Blood was drawn on days 3, 5 and 7 PI for determination of hematocrits, and serum electrolytes and osmolality. Hematocrits were determined by standard capillary tube techniques and serum electrolytes determined by flame photometer and a chloride analyzer (89). Serum osmolality was measured by osmometer (90). On day 7 PI, animals were killed. Fresh fecal matter was obtained from the terminal rectum and assessed for fecal water content. Ten cm of jejunal tissue was obtained starting 5 cm distal to the ligament of Treitz. The first cm of tissue was discarded and the second 2 cm fixed in 10% buffered formalin for histology (H&E). The next 1 cm of jejunal tissue was subsequently taken for mucosal pathogen counts. The remainder of the jejunal tissue was frozen for subsequent analysis of mucosal sucrase and maltase activity and protein content by established techniques (20). Jejunal villus height was determined by light microscopy (34) and mucosal wet weights assessed as previously described (20).

Statistical analysis: Statistical analysis was performed by analysis of variance (ANOVA) with a Tukey post-test. For analysis of the fecal scoring Dunn's post-test for non-parametric data was performed. P<0.05 was considered significant.

### Results

One control animal was excluded from the study due to the presence of bacterial colonies of unknown origin on MacConkey agar and one animal from the infected, ORS+EGF group was removed due to gavage injury. All infected animals were excreting the bacteria by day 3 and continued to do so for the remainder of the study.

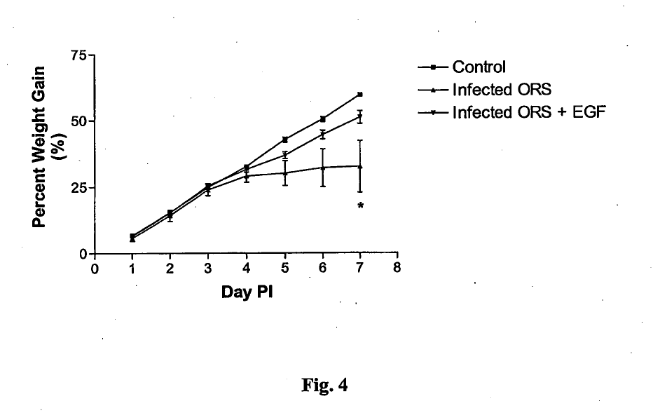

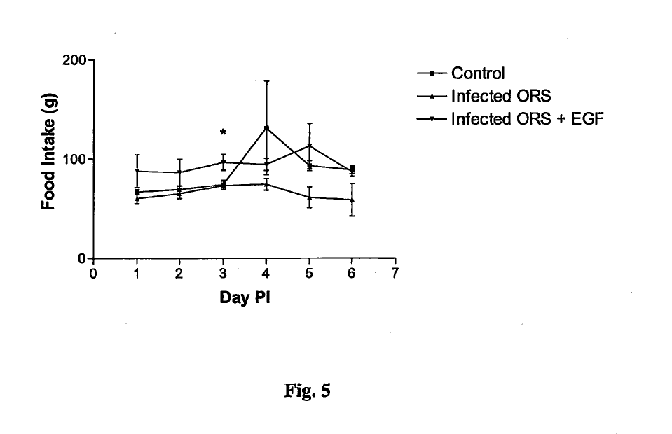

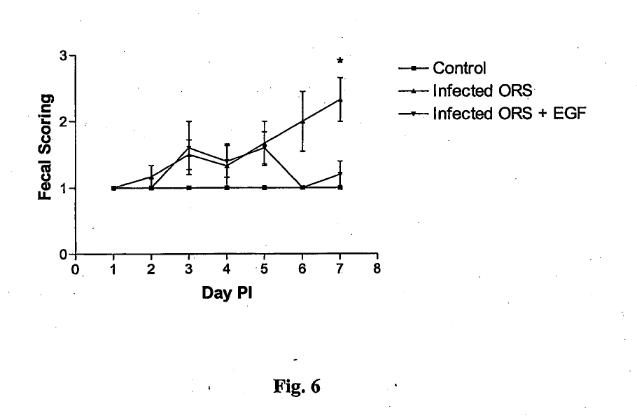

Clinical Assessment: Infected animals treated with ORS alone showed significantly (P>0.05) reduced weight gain expressed as a percentage of each animals original weight compared to control animals on day 7. Weight gain in animals treated with ORS+EGF did not differ from controls at any time point (FIG. 4). Food intake was greater in infected ORS+EGF treated animals compared to infected ORS treated animals throughout the study and greater than controls from day 1 to 3 of treatment but this effect only reached statistical significance on day 3 when food intake in infected ORS+EGF treated animals was greater than that measured in both infected ORS treated animals and controls (FIG. 5). As shown in FIG. 6 fecal scores were significantly increased (P<0.01) in infected ORS treated animals compared to control animals on day 7 of treatment. Fecal scores did not significantly differ between control and ORS+EGF treated animals.

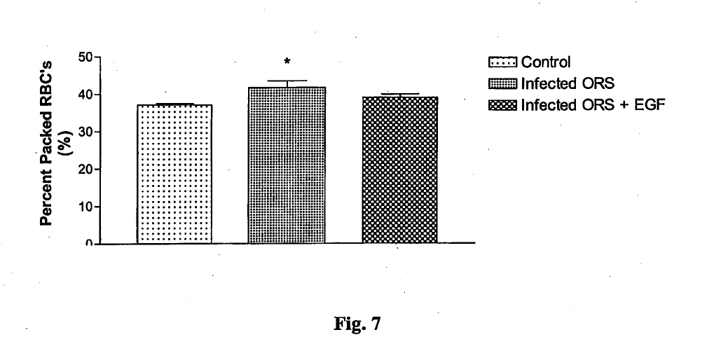

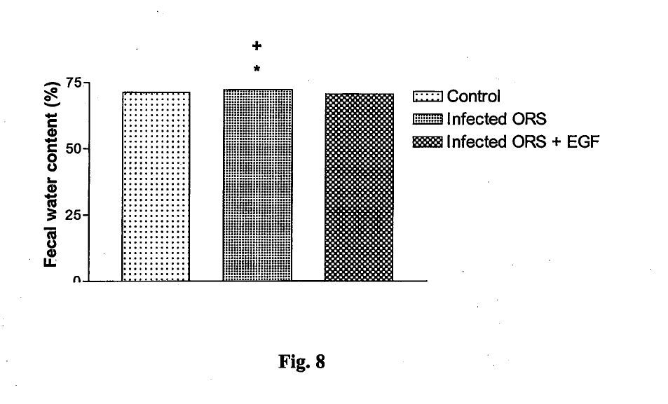

Dehydration and Diarrhea: Oral rehydration was also enhanced in infected animals treated with ORS+EGF compared to infected animals treated with ORS alone. FIG. 7 shows hematocrit values from blood obtained on day 5. Hematocrits were significantly increased (P<0.05) in infected ORS treated animals on day 5 compared to controls. Day 5 hematocrits of infected ORS+EGF treated animals did not differ from controls. No difference in hematocrit values was observed between the three groups on days 3 and 7. Likewise, no difference in serum chloride and sodium concentrations or serum osmolality was observed between the 3 groups at either day 3, 5 or 7. FIG. 8 shows fecal water content of fecal samples obtained from the rectum of animals at autopsy. Fecal water content was slightly but significantly increased (P<0.001) in infected ORS treated animals compared to controls. Fecal water content was significantly decreased (P<0.001) in infected ORS+EGF treated animals compared to infected animals treated with ORS alone.

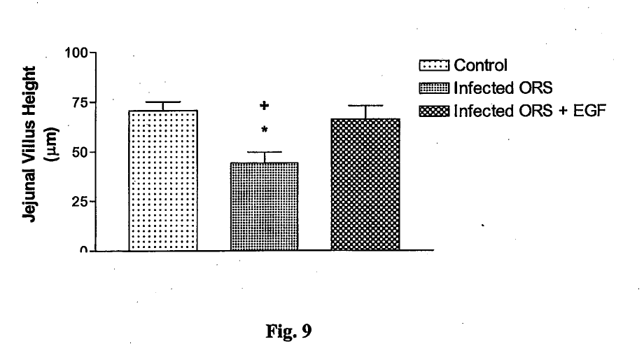

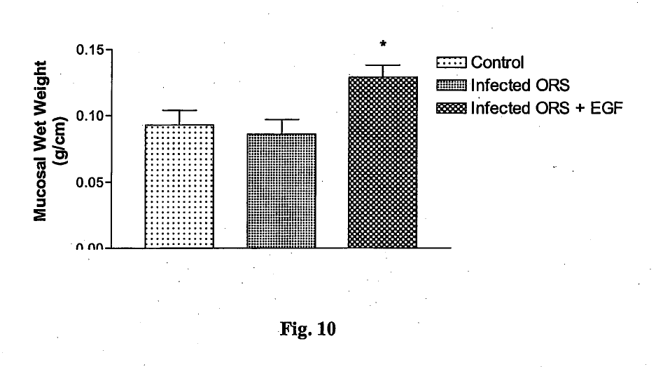

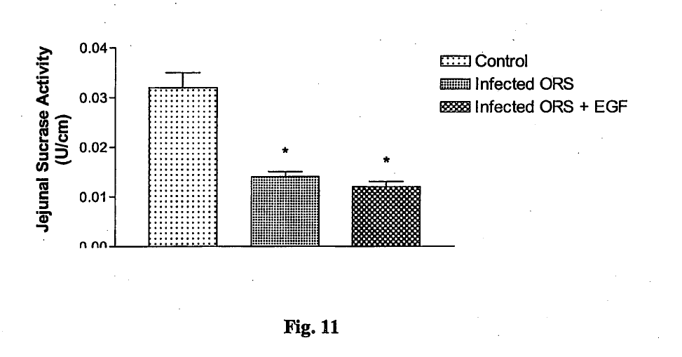

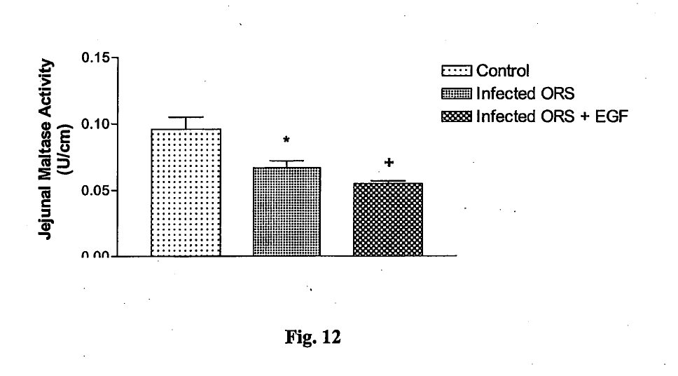

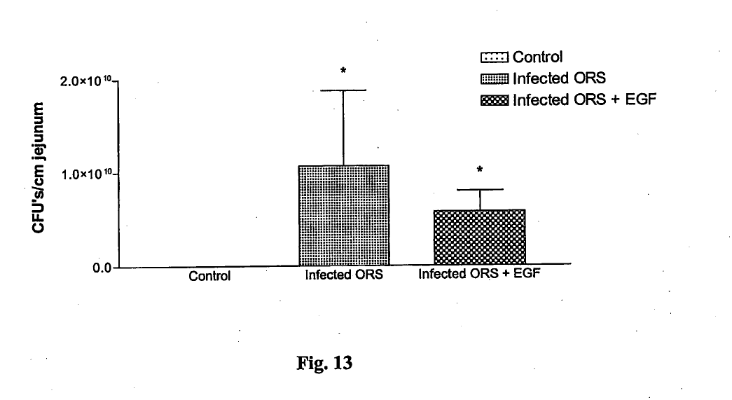

Histology, mucosal measurements and enzymes: Jejunal morphometric analysis was performed. FIG. 9 shows jejunal villus height in the three groups. Villus height was significantly (P<0.01) decreased in infected animals treated with ORS alone compared to control animals. Villus height was significantly (P<0.05) increased in infected animals treated with ORS+EGF compared to those given ORS alone. Villus height did not differ between control animals and infected animals treated with ORS+EGF. Crypt depth and villus width did not differ between any of the groups. FIG. 10 shows jejunal mucosal wet weight in control animals and infected animals treated with ORS or ORS+EGF. Mucosal wet weight was reduced in infected animals treated with ORS compared to controls but this effect did not reach significance. Mucosal wet weight was significantly (P<0.05) increased in infected animals treated with ORS+EGF compared to those given ORS alone. Mucosal wet weight did not statistically differ between control animals and infected animals treated with ORS+EGF. Mucosal protein content did not statistically differ between any of the groups (data not shown). Jejunal sucrase and maltase activity is shown in FIGS. 11 and 12, respectively. Jejunal sucrase and maltase activity was significantly reduced in infected animals treated with ORS or ORS+EGF when compared to controls. Sucrase and maltase activity did not differ between infected animals given either treatment. Jejunal mucosal bacterial counts were also significantly elevated in infected animals treated with ORS or ORS+EGF when compared to controls (FIG. 13). Though there was a trend towards decreased jejunal mucosal bacterial counts in the ORS+EGF group compared to the ORS treated group this effect did not reach statistical significance. However, a longer course of treatment would have likely resulted in a significant decrease in jejunal mucosal bacterial counts in the ORS+EGF group compared to the ORS treated group.

### Conclusions

Infected animals treated with ORS alone displayed reduced weight gain and significant diarrhea. Treating infected animals with ORS supplemented with EGF resulted in normalized weight gain and reduced severity and duration of diarrhea. ORS supplemented with EGF also resulted in enhanced oral rehydration and reduced fecal water content compared to infected animals treated with ORS alone. Lastly, infected animals treated with ORS alone showed a significant mucosal injury. Treating infected animals with ORS supplemented with EGF significantly improved mucosal healing compared to animals treated with ORS alone. While jejunal mucosal bacterial counts in the ORS+EGF group did not statistically differ from the ORS treated group, a longer course of treatment would have likely resulted in a significant decrease in jejunal mucosal bacterial counts in the ORS+EGF group compared to the ORS treated group. ORS supplemented with EGF provides significant clinical and histological benefit in the treatment of gastrointestinal infection, dehydration and diarrhea over treatment with ORS alone.

### Example 5

### Use of ORS Supplemented with EGF to Treat Diarrhea

These studies will utilize an established model of enteropathogenic E. coli infection in rabbits (20). Experiments will examine physiological parameters of rehydration and directly measure the ability of EGF supplemented ORS to stimulate water absorption from the lumen of the gut in infected animals.

### Methods

Weanling 4-week-old New Zealand White rabbits will be infected with a noninvasive, attaching effacing E. coli 015 (RDEC-1) obtained from the American Type Culture Collection. Briefly, experimental rabbits (400-600 g) will be orally inoculated with 5×10⁷ live E. coli in 1 mL 10% sodium bicarbonate (20). Controls will receive sodium bicarbonate only. Animals will be housed individually in a level B containment room, fed commercial feed and given water ad libitum. In one series of experiments, animals will be orally gavaged with 5 mls of standard ORS (Pedialyte™, Ross Products, Columbus, Ohio) supplemented with 100 ng/ml of recombinant human EGF (rhEGF) (Austral Biologicals, San Ramon, Calif.) or vehicle three times a day starting on day 3 postinfection (PI). Blood will be drawn on days 3, 5 and 7 PI for determination of hematocrits, and serum electrolytes and osmolality as previously described (32). In a second series of experiments, animals will be infected and in vivo small intestinal absorption of water, Na⁺, K⁺, and Cl⁻ examined on day 7 PI by single pass perfusion of standard ORS solutions supplemented with 100 ng/ml rhEGF or vehicle (61). Briefly, animals will be anesthetized and a 10 to 15 cm segment of jejunum, starting 10 cm distal to the ligament of Treitz, will be isolated and cannulated at each end. Standard ORS (Pedialyte™, Ross Products, Columbus, Ohio) supplemented with 100 ng/ml of rhEGF (Austral Biologicals, San Ramon, Calif.) or vehicle will then be perfused through the segment at a constant rate of 0.1 ml/min. Five g/l of polyethylene glycol (PEG) 4000 and 10 μCi/l of [¹⁴C] PEG will be added to all solutions as the non absorbable marker. After an initial 60-minute equilibration period using standard ORS, three consecutive 20-minute perfusate samples will be collected from the distal site. The perfusate will then be changed to include 100 ng/ml rhEGF and after a 10 minute period to allow the EGF solution to pass through the loop, a further six consecutive 10 minute samples will be collected. Absorption of Na⁺, K⁺ will be determined by flame spectrophotometry, Cl⁻ by chloride analyzer and water absorption determined by measuring the concentration of the non-absorbable marker [¹⁴C] polyethylene glycol by scintillation spectrometry (59).

### Results

Rehydration will be enhanced in infected animals treated with EGF supplemented ORS compared to those receiving ORS alone. Hematocrits, serum electrolytes and serum osmolality will all be improved in animals treated with EGF supplemented ORS compared to those receiving ORS alone. Finally, absorption of water and sodium will be significantly increased in animals perfused with EGF supplemented ORS compared to those perfused with ORS alone.

### Conclusions

It is expected that EGF supplemented ORS will significantly improve oral rehydration in an animal model of established diarrhea over standard ORS alone.

### Example 6

### Use of ORS Supplemented with EGF to Reduce the Severity and Duration of Diarrhea

These studies will examine the ability of EGF supplemented ORS to reduce the severity and duration of diarrhea in an established model of enteropathogenic E. coli infection in rabbits (20).

### Methods

Weanling 4-week-old New Zealand White rabbits will be infected with a noninvasive, attaching effacing E. coli 015 (RDEC-1) obtained from the American Type Culture Collection. Briefly, experimental rabbits (400-600 g) will be orally inoculated with 5×10⁷ live E. coli in 1 mL 10% sodium bicarbonate (20). Controls will receive sodium bicarbonate only. Animals will be housed individually in a level B containment room, fed commercial feed and given water ad libitum. All animals will be orally gavaged with 5 mls of standard ORS (Pedialyte™, Ross Products, Columbus, Ohio) supplemented with 100 ng/ml of rhEGF (Austral Biologicals, San Ramon, Calif.) or vehicle three times a day starting on day 3 postinfection (PI). Weight gain and food intake will be measured, fecal water content determined and fecal scoring obtained on a four point scale (41), and pathogen counts assessed from rectal swabs plated onto MacConkey agar (Difco Laboratories, Detroit, Mich.) daily (20).

### Results

Treatment with EGF supplemented ORS is anticipated to improve weight gain, and decrease fecal water content and fecal scoring, and pathogen counts in infected animals compared to infected animals treated with standard ORS alone.

### Conclusions

It is expected that EGF supplemented ORS will significantly reduce the severity and duration of diarrhea in an animal model of established diarrhea over ORS alone.

### Example 7

### Use of ORS Supplemented with EGF to Promote Intestinal Healing

Studies will examine intestinal damage in an established model of enteropathogenic E. coli infection in rabbits. Intestinal healing will be assessed in infected animals treated with standard ORS compared to infected animals treated with standard ORS supplemented with EGF.

### Methods

Weanling 4-week-old New Zealand White rabbits will be infected with a noninvasive, attaching effacing E. coli 015 (RDEC-1) obtained from the American Type Culture Collection. Briefly, experimental rabbits (400-600 g) will be orally inoculated with 5×10⁷ live E. coli in 1 mL 10% sodium bicarbonate (20). Controls will receive sodium bicarbonate only. Animals will be housed individually in a level B containment room, fed commercial feed and given water ad libitum. All animals will be orally gavaged with 5 mls of standard ORS (Pedialyte™, Ross Products, Columbus, Ohio) supplemented with 100 ng/ml of rhEGF (Austral Biologicals, San Ramon, Calif.) or vehicle three times a day starting on day 3 postinfection (PI). Mucosal permeability will be assessed on day 4 and 6 PI by oral gavage of 90 mg Cr-EDTA and subsequent measurement in the urine as previously reported (41). On day 7 PI animals will be killed and 10 cm of small intestinal tissue obtained starting from 10 cm distal to the ligament of Treitz and 10 cm proximal to the ileocecal valve for light (35) and transmission electron microscopy (20) and measurement of mucosal sucrase and maltase activity by established techniques (20).

### Results

Mucosal permeability is predicted to decrease in animals treated with EGF supplemented ORS on day 6 PI compared to animals treated with standard ORS. In addition, treatment with EGF supplemented ORS will improve histological damage and sucrase and maltase activity in the small intestine of infected animals over that seen in infected animals treated with ORS alone.

### Conclusions

It is expected that EGF supplemented ORS will significantly improve mucosal permeability, lactase activity and intestinal damage in an animal model of established diarrhea over standard ORS alone.

### Example 8

### Use of EGF in an Oral Composition to Enhance Oral Rehydration, Reduce the Severity and Duration of Diarrhea and Promote Intestinal Healing

Studies will examine the use of EGF in an oral composition to enhance oral rehydration, reduce the severity and duration of diarrhea and promote intestinal healing in an established model of enteropathogenic E. coli infection in rabbits.

### Methods

Weanling 4-week-old New Zealand White rabbits will be infected with a noninvasive, attaching effacing E. coli 015 (RDEC-1) obtained from the American Type Culture Collection. Briefly, experimental rabbits (400-600 g) will be orally inoculated with 5×10⁷ live E. coli in 1 mL 10% sodium bicarbonate (20). Controls will receive sodium bicarbonate only. Animals will be housed individually in a level B containment room, fed commercial feed and given water ad libitum. All animals will be orally gavaged with 5 mls of water supplemented with 100 ng/ml of rhEGF (Austral Biologicals, San Ramon, Calif.) and 10 mg/ml of casein as a bystander protein or vehicle (water, containing 10 mg/ml casein) three times a day starting on day 3 postinfection (PI). Mucosal permeability will be assessed on day 4 and 6 PI by oral gavage of 90 mg Cr-EDTA and subsequent measurement in the urine as previously reported (41). On day 7 PI animals will be killed and 10 cm of small intestinal tissue obtained starting from 10 cm distal to the ligament of Treitz and 10 cm proximal to the ileocecal valve for light (35) and transmission electron microscopy (20) and measurement of mucosal sucrase and maltase activity by established techniques (20). Weight gain and food intake will be measured, fecal water content determined and fecal scoring obtained on a four point scale (41), and pathogen counts assessed from rectal swabs plated onto MacConkey agar (Difco Laboratories, Detroit, Mich.) daily (20). In a second series of experiments, animals will be infected and treated as above and in vivo small intestinal absorption of water, Na⁺, K⁺, and Cl⁻ examined on day 7 PI by single pass perfusion of standard ORS solutions (61). Briefly, animals will be anesthetized and a 10 to 15 cm segment of jejunum, starting 10 cm distal to the ligament of Treitz, will be isolated and cannulated at each end. Standard ORS (Pedialyte™, Ross Products, Columbus, Ohio) will then be perfused through the segment at a constant rate of 0.1 ml/min. Five g/l of polyethylene glycol (PEG) 4000 and 10 μCi/1 of [¹⁴C] PEG will be added to all solutions as the non absorbable marker. After an initial 60-minute equilibration period, three consecutive 20-minute perfusate samples will be collected from the distal site. Absorption of Na⁺, K⁺ will be determined by flame spectrophotometry, Cl⁻ by chloride analyzer and water absorption determined by measuring the concentration of the non-absorbable marker [¹⁴C] polyethylene glycol by scintillation spectrometry.

### Results

Mucosal permeability is anticipated to decrease in animals treated with the oral EGF composition on day 6 PI compared to animals treated with vehicle. In addition, treatment with the oral EGF composition will improve histological damage and sucrase and maltase activity in the small intestine, improve weight gain, decrease fecal water content, fecal scoring and pathogen counts and improve absorption of water and sodium from the small intestine of infected animals compared to animals receiving vehicle alone.

### Conclusions

It is expected that the oral EGF composition will significantly improve mucosal permeability, lactase activity, intestinal damage, severity and duration of diarrhea, and the absorption of sodium and water in an animal model of established diarrhea over vehicle alone.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents, and applications are herein incorporated by reference in their entirety to the same extent as if each individual publication, patent or application was specifically and individually indicated to be incorporated by reference in its entirety.

### REFERENCES

1. Alpers, D. H. Digestion and absorption of carbohydrates and proteins. In Johnson, L. R., ed. Physiology of the Gastrointestinal Tract. New York, Raven Press. 1994, 1723-1750.
2. Bahl, R., N. Bhandari, M. Saksena, T. Strand, G. T. Kumar, M. K. Bhan, and H. Sommerfelt. Efficacy of zinc-fortified oral rehydration solution in 6- to 35-month-old children with acute diarrhea. J. Pediatr. 141: 677-682, 2002.
3. Beubler, E. and A. Schirgi-Degen. Nitric oxide counteracts 5-hydroxytryptamine- and cholera toxin-induced fluid secretion and enhances the effect of oral rehydration solution. Eur. J. Pharmacol. 326: 223-228, 1997.
4. Bhan, M. K., O. P. Ghai, V. Khoshoo, A. S. Vasudev, S. Bhatnagar, N. K. Arora, Rashmi, and G. Stintzing. Efficacy of mung bean (lentil) and pop rice based rehydration solutions in comparison with the standard glucose electrolyte solution. J. Pediatr. Gastroenterol. Nutr. 6: 392-399, 1987.
5. Bhan, M. K., D. Mahalanabis, O. Fontaine, and N. F. Pierce. Clinical trails of improved oral rehydration salt formulations: a review. Bull. W.H.O. 72: 945-955, 1994.
6. Bhan, M. K., S. Sazawal, S. Bhatnagar, N. Bhandari, D. K. Guha, and S. K. Aggarwal. Glycine, glycyl-glycine and maltodextrin based oral rehydration solution: assessment of efficacy and safety in comparison to standard ORS. Acta Paediatr. Scand. 79: 518-526, 1990.
7. Bhatnagar, S., R. Bahl, P. K. Sharma, G. T. Kumar, S. K. Saxena, and M. K. Bhan. Zinc with oral rehydration therapy reduces stool output and duration of diarrhea in hospitalized children: a randomized controlled trial. J. Pediatr. Gastroenterol. Nutr. 38: 34-40, 2004.
8. Bhutta, Z. A., S. M. Bird, R. E. Black, K. H. Brown, J. M. Gardner, A. Hidayat, F. Khatun, R. Martorell, N. X. Ninh, M. E. Penny, J. L. Rosado, S. K. Roy, M. Ruel, S. Sazawal, and A. Shankar. Therapeutic effects of oral zinc in acute and persistent diarrhea in children in developing countries: pooled analysis of randomized controlled trials. Am. J. Clin. Nutr. 72: 1516-1522, 2000.
9. Bird, A. R., W. J. Croom, Jr., Y. K. Fan, L. R. Daniel, B. L. Black, B. W. McBride, E. J. Eisen, L. S. Bull, and I. L. Taylor. Jejunal glucose absorption is enhanced by epidermal growth factor in mice. J. Nutr. 124: 231-240, 1994.
10. Boonstra, J., P. Rijken, B. Humbel, F. Cremers, A. Verkleij, and P. Van Bergen en Henegouwen. The epidermal growth factor. Cell Biol. Int. 19: 413-430, 1995.
11. Buret, A., D. G. Gall, and M. E. Olson. Effects of murine giardiasis on growth, intestinal morphology and disaccharidase activity. J. Parasitol. 76: 403-409, 1990.
12. Buret, A., Gall, D. G., Olson, M. E., and Hardin, J. A. Epidermal growth factor (EGF) prevents Escherichia Coli enteritis in rabbits and bacterial translocation in vitro. 7th International Congress for Infectious Disease. 1996.
13. Buret, A., Gall, D. G., Olson, M. E., and Hardin, J. A. Prevention of enteric infections by epidermal growth factor (EGF). 36th Interscience Conference on Antimicrobial Agents and Chemotherapy, New Orleans, La. 1996.
14. Buret, A., Gall, D. G., Olson, M. E., and Hardin, J. A. Anti-infective properties of a mucosal cytokine: epidermal growth factor (EGF). Proceedings of the International Biofilm Symposium, Canmore, Alberta. 1997.
15. Buret, A., D. G. Gall, M. E. Olson, and J. A. Hardin. The role of the epidermal growth factor receptor in microbial infections of the gastrointestinal tract. Microbe. Infect. 1: 1139-1144, 1999.
16. Buret, A., Hardin, J. A., Olson, M. E., Chin, A., and Gall, D. G. Effects of orally administered epidermal growth factor (EGF) during Escherichia coli infection in rabbits. Gastroenterology 110, A793. 1996.
17. Buret, A., J. A. Hardin, M. E. Olson, and D. G. Gall. Pathophysiology of small intestinal malabsorption in gerbils infected with Giardia lamblia. Gastroenterology 103: 506-513, 1992.
18. Buret, A., Kamieniecky, D., Olson, M. E., Gall, D. G., and Hardin, J. A. Epithelial colonization by Cryptosporidium and trans-epithelial electrical resistance: effects of epidermal growth factor (EGF). Gastroenterology 116, A865. 1999.
19. Buret, A., E. V. O'Loughlin, G. H. Curtis, and D. G. Gall. Effect of acute Yersinia enterocolitica infection on small intestinal ultrastructure. Gastroenterology 98: 1401-1407, 1990.
20. Buret, A., M. E. Olson, D. G. Gall, and J. A. Hardin. Effects of orally administered epidermal growth factor on enteropathogenic Escherichia coli infection in rabbits. Infect. Immun. 66: 4917-4923, 1998.
21. Buret, A., Olson, M. E., Gall, D. G., Hardin, J. A., Kamieniecky, D., and Lupul, S. Epidermal growth factor (EGF) inhibits intestinal colonization with Cryptosporidium parvum. Arch. Pharmacol. 358(Suppl. 1), 8359. 1998.
22. Buret, A. G., Chin, A. C., and Scott, K. G. E. Infection of human and bovine epithelial cells with Cryptosporidium andersoni induces apoptosis and disrupts tight-junctional ZO-1: effects of epidermal growth factor. Int. J. Parasitol. 33, 1363-1371. 2003.
23. Carneiro-Filho, B. A., O. Y. Bushen, G. A. Brito, A. A. Lima, and R. L. Guerrant. Glutamine analogues as adjunctive therapy for infectious diarrhea. Curr. Infect. Dis. Rep. 5: 114-119, 2003.
24. Chang, E. B. Intestinal water and electrolyte absorption and secretion. Transplant. Proc. 28: 2679-2682, 1996.
25. Chung, B. M., Wallace, L. E., Hardin, J. A., and Gall, D. G. Epidermal growth factor increases rabbit jejunal glucose transport by recruiting an intracellular pool of SGLT1 into the brush border. Can. J. Gastroenterol. 13, A205. 1999.
26. Chung, B. M., J. K. Wong, J. A. Hardin, and D. G. Gall. SGLT1 expression and the role of actin in EGF-induced alterations in enterocyte membrane function and surface area. Am. J. Physiol. (Gastrointest. Liver Physiol.) 276: G463-G469, 1999.
27. Cohen, M. B., A. G. Mezoff, D. W. Jr. Laney, J. A. Bezerra, B. M. Beane, D. Drainer, R. Baker, and J. R. Moran. Use of a single solution for oral rehydration and maintenance therapy of infants with diarrhea and mild to moderate dehydration. Pediatrics 95: 639-645, 1995.
28. Dahms, N. M. and R. L. Schnaar. Ganglioside composition is regulated during differentiation in the neuroblastoma X glioma hybrid cell line NG108-15. J. Neurosci. 3: 806-817,1983.
29. Donowitz, M., J. L. M. Montgomery, M. S. Walker, and M. E. Cohen. Brush-border tyrosine phosphorylation stimulates ileal neutral NaCl absorption and brush-border Na⁺-H⁺ exchange. Am. J. Physiol. Gastrointest. Liver Physiol. 266: G647-G656, 1994.
30. Elliott, S, N., McKnight, W., Gall, D. G., Hardin, J. A., Olson, M. E., Wallace, J. L., and Buret, A. Epidermal growth factor (EGF)-induced gastric ulcer healing is independent of a bactericidal action. Mediators of Inflammation 8(Suppl. 1), S81. 1999.
31. Elliott, S, N., J. L. Wallace, W. McKnight, D. G. Gall, J. A. Hardin, M. Olson, and A. Buret. Bacterial colonization and healing of gastric ulcers: the effects of epidermal growth factor. Am. J. Physiol Gastrointest. Liver Physiol 278: G105-G112, 2000.
32. Garthwaite, B. D., J. K. Drackley, G. C. McCoy, and E. H. Jaster. Whole milk and oral rehydration solution for calves with diarrhea of spontaneous origin. J. Dairy Sci. 77: 835-843, 1994.
33. Gibbs, S., A. N. Silva Pinto, S. Murli, M. Huber, D. Hohl, and M. Ponec. Epidermal growth factor and keratinocyte growth factor differentially regulate epidermal migration, growth, and differentiation. Wound Repair Regen. 8: 192-203, 2000.
34. Hardin, J. A., A. Buret, J. B. Meddings, and D. G. Gall. Effect of epidermal growth factor on enterocyte brush-border surface area. Am. J. Physiol. 264: G312-G318, 1993.
35. Hardin, J. A., L. Donegan, B. Chung, J. Fung, and D. G. Gall. Intestinal adaptation in the spontaneously diabetic BB rat. Diabetes Res. 34: 125-138, 1999.
36. Hardin, J. A. and D. G. Gall. The effect of TGFD on intestinal solute transport. Reg. Pep. 39: 169-176, 1992.
37. Hardin, J. A., Olson, M. E., Buret, A. G., and Gall, D. G. The effect of oral EGF on Giardiasis in gerbils. Gastroenterology 112, A992. 1997.
38. Hardin, J. A., J. K. Wong, C. L Cheeseman, and D. G. Gall. The effect of luminal epidermal growth factor on enterocyte glucose and proline transport. Am. J. Physiol. Gastrointest. Liver Physiol. 271: G509-G515, 1996.
39. Holtug, K., M. B. Hansen, and E. Skadhauge. Experimental studies of intestinal ion and water transport. Scand. J. Gastroenterol. 31 Suppl. 216: 95-110, 1996.
40. Horváth, K., I. D. Hill, P. Devarajan, D. Mehta, S. C. Thomas, R. B. Lu, and E. Lebenthal. Short-term effect of epidermal growth factor (EGF) on sodium and glucose cotransport of isolated jejunal epithelial cells. Biochim. Biophys. Acta Mol. Cell Res. 1222: 215-222, 1994.
41. Hunt, E., Q. Fu, M. U. Armstrong, D. K. Rennix, D. W. Webster, J. A. Galanko, W. Chen, E. M. Weaver, R. A. Argenzio, and J. M. Rhoads. Oral bovine serum concentrate improves cryptosporidial enteritis in calves. Pediatr. Res. 51: 370-376, 2002.
42. Hunt, J. B., E. J. Elliot, P. D. Fairclough, M. L. Clark, and M. J. Farthing. Water and solute absorption from hypotonic glucose-electrolyte solutions in human jejunum. Gut 33: 479-483, 1992.
43. Islam, S., D. Mahalanabis, A. K. Chowdhury, M. A. Wahed, and A. S. Rahman. Glutamine is superior to glucose in stimulating water and electrolyte absorption across rabbit ileum. Dig. Dis. Sci. 42: 420-423, 1997.
44. Jodal, M. and Lundgren, O. Intestinal water transport. Acta Paediatr. Scand. 305, 49-55. 1983.
45. Jorgensen, P. E., L. G. Jensen, B. S. Sorensen, S. S. Poulsen, and E. Nexo. Pig epidermal growth factor precursor contains segments that are highly conserved among species. Scand. J. Clin. Lab. Invest. 58: 287-297, 1998.
46. Lenferink, A. E., E. J. Van Zoelen, M. J. Van Vugt, S. Grothe, W. van Rotterdam, M. L. Van de Poll, and M. D. O'Connor-McCourt. Superagonistic activation of ErbB-1 by EGF-related growth factors with enhanced association and dissociation rate constants. J. Biol. Chem. 275: 26748-26753, 2000.
47. Lima, A. A., G. H. Carvalho, A. A. Figueiredo, A. R. Gifoni, A. M. Soares, E. A. Silva, and R. L. Guerrant. Effects of an alanyl-glutamine-based oral rehydration and nutrition therapy solution on electrolyte and water absorption in a rat model of secretory diarrhea induced by cholera toxin. Nutrition 18: 458-462, 2002.
48. Liu, C. D., A. J. Rongione, M. S. Shin, S. W. Ashley, and D. W. McFadden. Epidermal growth factor improves intestinal adaptation during somatostatin administration in vivo. J. Surg. Res. 63: 163-168, 1996.
49. Loo, D. D., B. A. Hirayama, A. K. Meinild, G. Chandy, T. Zeuthen, and E. M. Wright. Passive water and ion transport by cotransporters. J. Physiol. 518: 195-202, 1999.
50. Loo, D. D. F., T. Zeuthen, G. Chandy, and E. M. Wright. Cotransport of water by the Na⁺/glucose cotransporter. Proc. Natl. Acad. Sci. USA 93: 13367-13370, 1996.
51. Ma, T. and A. S. Verkman. Aquaporin water channels in gastrointestinal physiology. J. Physiol. 517: 317-326, 1999.
52. Masyuk, A. I., R. A. Marinelli, and N. F. LaRusso. Water transport by epithelia of the digestive tract. Gastroenterology 122: 545-562, 2002.
53. Maulen-Radovan, I., P. Gutierrez-Castrellon, M. Hashem, M. Neylan, G. Baggs, R. Zaldo, L. I. Ndife, P. F. Pollack, and M. Santosham. Safety and efficacy of a premixed, rice-based oral rehydration solution. J. Pediatr. Gastroenterol. Nutr. 38: 159-163, 2004.
54. Mehta, D. I., K. Horvath, S. Chanasongcram, I. D. Hill, and P. Panigrahi. Epidermal growth factor up-regulates sodium-glucose cotransport in enterocyte models in the presence of cholera toxin. J. Parent. Enter. Nutr. 21: 185-191, 1997.
55. Nalin, D. R. Oral replacement of water and electrolyte losses due to traveller's diarrhea. Scand. J. Gastroenterol. 84: S95-S98, 1983.
56. Nappert, G., J. M. Barrios, G. A. Zello, and J. M. Naylor. Oral rehydration solution therapy in the management of children with rotavirus diarrhea. Nutr. Rev. 58: 80-87, 2000.
57. Nexo, E. and H. F. Hansen. Binding of epidermal growth factor from man, rat and mouse to the human epidermal growth factor receptor. Biochim. Biophys. Acta 843: 101-106, 1985.
58. O'Loughlin, E. V., D. G. Gall, and C. H. Pai. Yersinia enterocolitica: Mechanisms of microbial pathogenesis and pathophysiology of diarrhoea. J. Gastroenterol. Hepatol. 5: 173-179, 1990.
59. O'Loughlin, E. V., C. H. Pai, and D. G. Gall. Effect of acute Yersinia enterocolitica infection on in vivo and in vitro small intestinal solute and fluid absorption in the rabbit. Gastroenterology 94: 664-672, 1988.
60. O'Loughlin, E. V., R. B. Scott, and D. G. Gall. Pathophysiology of infectious diarrhea:changes in intestinal structure and function. J. Pediatr. Gastroenterol. Nutr. 12: 5-20, 1991.
61. Opleta-Madsen, K., J. Hardin, and D. G. Gall. Epidermal growth factor upregulates intestinal electrolyte and nutrient transport. Am. J. Physiol. 260: G807-G814, 1991.
62. Pascall, J. C., D. S. C. Jones, S. M. Doel, J. M. Clements, M. Hunter, T. Fallon, M. Edwards, and K. D. Brown. Cloning and characterization of a gene encoding pig epidermal growth factor. J. Mol. Endocrinol. 6: 63-70, 1991.
63. Patel, A. B., L. A. Dhande, and M. S. Rawat. Economic evaluation of zinc and copper use in treating acute diarrhea in children: a randomized controlled trial. Cost Effectiveness and Resource Allocation 1: 7, 2003.
64. Piazuelo, E., P. Jimenez, A. Lanas, A. Garcia, F. Esteva, and R. Sainz. Platelet-derived growth factor and epidermal growth factor play a major role in human colonic fibroblast repair activities. Eur. Surg. Res. 32: 191-196, 2000.
65. Playford, R. J., A. C. Woodman, P. Clark, P. Watanapa, D. Vesey, P. H. Deprez, R. C. N. Williamson, and J. Calam. Effect of luminal growth factor preservation on intestinal growth. Lancet 341: 843-848, 1993.
66. Rao, R. K., D. W. Thomas, S. Pepperl, and F. Porreca. Salivary epidermal growth factor plays a role in protection of ileal mucosal integrity. Dig. Dis. Sci. 42: 2175-2181, 1997.
67. Rautanen, T., E. Isolauri, E. Salo, and T. Vesikari. Management of acute diarrhoea with low osmolarity oral rehydration solutions and Lactobacillus strain GG. Arch. Dis. Child. 79: 157-160, 1998.
68. Rhoads, J. M., G. G. Gomez, W. Chen, R. Goforth, R. A. Argenzio, and M. J. Neylan. Can a super oral rehydration solution stimulate intestinal repair in acute viral enteritis? J. Diarrhoeal Dis. Res. 14: 175-181, 1996.
69. Rhoads, J. M., R. J. MacLeod, and J. R. Hamilton. Alanine enhances jejunal sodium absorption in the presence of glucose: studies in piglet viral diarrhea. Pediatr. Res. 20: 879-883, 1986.
70. Rhoads, M. Management of acute diarrhea in infants. J. Parent. Enter. Nutr. 23: S18-S19, 1999.
71. Rongione, A. J., A. M. Kusske, T. R. Newton, S. W. Ashley, M. J. Zinner, and D. W. McFadden. EGF and TGF stimulate proabsorption of glucose and electrolytes by Na⁺/glucose cotransporter in awake canine model. Dig. Dis. Sci. 46: 1740-1747, 2001.
72. Rongione, A. J., C. D. Liu, E. E. Whang, J. C. Y. Dunn, T. R. Newton, M. J. Zinner, S. W. Ashley, and D. W. McFadden. Epidermal growth factor enhances small intestinal absorption. Surgical Forum 46: 188-190, 1995.
73. Salloum, R. M., B. R. Stevens, G. S. Schultz, and W. W. Souba. Regulation of small intestinal glutamine transport by epidermal growth factor. Surgery 113: 552-559, 1993.
74. Sazawal, S., S. Bhatnagar, M. K. Bhan, S. K. Saxena, N. K. Arora, S. K. Aggarwal, and D. K. Kashyap. Alanine-based oral rehydration solution: assessment of efficacy in acute noncholera diarrhea among children. J. Pediatr. Gastroenterol. Nutr. 12: 461-468, 1991.
75. Seydel, A. S., J. H. Miller, T. P. Sarac, C. K. Ryan, W. Y. Chey, and H. C. Sax. Octreotide diminishes luminal nutrient transport activity, which is reversed by epidermal growth factor. Am. J. Surg. 172: 267-271, 1996.
76. Shwartz, M. Z. and R. B. Storozuk. Influence of epidermal growth factor on intestinal function in the rat: comparison of systemic infusion versus luminal perfusion. Am. J. Surg. 155: 18-22, 1988.
77. Simpson, R. J., J. A. Smith, R. L. Moritz, M. J. O'Hare, P. S. Rudland, J. R. Morrison, C. J. Lloyd, B. Grego, A. W. Burgess, and E. C. Nice. Rat epidermal growth factor: complete amino acid sequence. Eur. J. Biochem. 153: 629-637, 1985.
78. Subbotina, M. D., V. N. Timchenko, M. M. Vorobyov, Y. S. Konunova, A. S. Aleksandrovih, and S. Shushunov. Effect of oral administration of tormentil root extract (Potentilla tormentilla) on rotavirus diarrhea in children: a randomized, double blind, controlled study. Pediatr. Infect. Dis. J. 22: 706-711, 2003.
79. Takano, J. and J. H. Yardley. Jejunal lesions in patients with giardiasis and malabsorption. An electron microscopic study. Bull. Johns Hopk. Hosp. 116: 413-429, 1965.
80. Taki, T., M. Abe, and M. Matsumoto. Simple purification method for anti-glycolipid antibody using polystyrene latex beads coated with gangliotetraosylceramide. J. Biochem. (Tokyo) 91: 1813-1816, 1982.
81. Tiemeyer, M., Y. Yasuda, and R. L. Schnaar. Ganglioside-specific binding protein on rat brain membranes. J. Biol. Chem. 264: 1671-1681, 1989.
82. Tonb, D., R. Mehta, H. Wang, J. Tung, and D. I. Mehta. Short-term effect of epidermal growth factor on glucose uptake in endoscopic biopsies. Dig. Dis. Sci. 48: 1614-1618, 2003.
83. Vesikari, T. and E. Isolauri. Glycine supplemented oral rehydration solutions for diarrhoea. Arch. Dis. Child. 61: 372-376, 1986.
84. VIIIa, X., J. W. Kuluz, C. L. Schleien, and J. F. Thompson. Epidermal growth factor reduces ischemia-reperfusion injury in rat small intestine. Crit. Care Med. 30: 1576-1580, 2002.
85. Wapnir, R. A., M. A. Wingertzahn, J. Moyse, and S. Teichberg. Gum arabic promotes rat jejunal sodium and water absorption from oral rehydration solutions in two models of diarrhea. Gastroenterology 112: 1979-1985, 1997.
86. Wingertzahn, M. A., S. Teichberg, and R. A. Wapnir. Modified starch enhances absorption and accelerates recovery in experimental diarrhea in rats. Pediatr. Res. 45: 397-402, 1999.
87. Wright, E. M., B. A. Hirayama, D. D. F. Loo, E. Turk, and K. Hagar. Intestinal sugar transport. In Johnson, L. R., ed. Physiology of the gastrointestinal tract. New York, Raven Press. 1994, 1751-1772.
88. Alam, N. H., R. N. Majumder, and G. J. Fuchs. Efficacy and safety of oral rehydration solution with reduced osmolarity in adults with cholera: a randomized double-blind clinical study. Lancet 354: 296-299, 1999.
89. Catto-Smih, A. G., J. Hardin, M. Patrick, E. V. O'Loughlin, and D. G. Gall. The effect or atrial natriuretic factor on intestinal electrolyte transport. Reg. Pep. 36: 29-44, 1991.
90. Pensyl, C. D., Benjamin, W. J. Vapor pressure osmometry: minimum sample microvolumes. Acta Ophthalmol. Scand. 77(1): 27-30, 1999.

What is claimed is:

1. Use of an oral composition comprising an epidermal growth factor (EGF), an epidermal growth factor receptor agonist, or a pharmaceutically acceptable salt form thereof for treating diarrhea.

2. Use of an oral composition comprising an epidermal growth factor (EGF), an epidermal growth factor receptor agonist, or a pharmaceutically acceptable salt form thereof for reducing the severity of diarrhea.

3. Use of an oral composition comprising an epidermal growth factor (EGF), an epidermal growth factor receptor agonist, or a pharmaceutically acceptable salt form thereof for reducing the duration of diarrhea.

4. Use of an oral composition comprising an epidermal growth factor (EGF), an epidermal growth factor receptor agonist, or a pharmaceutically acceptable salt form thereof for promoting healing of intestinal damage associated with diarrhea.

5. Use of an oral composition comprising an epidermal growth factor (EGF), an epidermal growth factor receptor agonist, or a pharmaceutically acceptable salt form thereof for treating dehydration associated with diarrhea.

6. Use of an oral composition comprising an epidermal growth factor (EGF), an epidermal growth factor receptor agonist, or a pharmaceutically acceptable salt form thereof for reducing bacterial colonization in an established diarrhea infection.

7. Use of an oral composition comprising an epidermal growth factor (EGF), an epidermal growth factor receptor agonist, or a pharmaceutically acceptable salt form thereof for reducing weight loss in an animal having diarrhea.

8. Use of an oral composition comprising an epidermal growth factor (EGF), an epidermal growth factor receptor agonist, or a pharmaceutically acceptable salt form thereof for increasing food uptake in an animal having diarrhea.

9. Use of an oral composition comprising an epidermal growth factor (EGF), an epidermal growth factor receptor agonist, or a pharmaceutically acceptable salt form thereof for enhancing rehydration in an animal having diarrhea.

10. Use of an oral composition comprising an epidermal growth factor (EGF), an epidermal growth factor receptor agonist, or a pharmaceutically acceptable salt form thereof for reducing water content in fecal matter in an animal having diarrhea.

11. Use of an oral composition comprising an epidermal growth factor (EGF), an epidermal growth factor receptor agonist, or a pharmaceutically acceptable salt form thereof for increasing villus height in an animal having diarrhea.

12. Use of an oral composition comprising an epidermal growth factor (EGF), an epidermal growth factor receptor agonist, or a pharmaceutically acceptable salt form thereof for improving mucosal healing in an animal having diarrhea.

13. Use of an oral composition comprising an epidermal growth factor (EGF), an epidermal growth factor receptor agonist, or a pharmaceutically acceptable salt form thereof for enhancing mucosal wet weight in an animal having diarrhea.

14. The use of any of claims 1 to 13 wherein the diarrhea is infectious malabsorptive diarrhea.

15. The use of any of claims 1 to 13 wherein the diarrhea is neonatal diarrhea.

16. The use of any of claims 1 to 13 wherein the diarrhea is secretory diarrhea.

17. The use of any of claims 1 to 16 wherein the composition is an ORS.

18. The use of the oral composition of any of claims 1 to 17 wherein the composition comprises an epidermal growth factor.

19. The use of the oral composition of claim 18 wherein the epidermal growth factor is a human epidermal growth factor.

20. The use of the oral composition of any of claims 1 to 17 wherein the composition comprises an epidermal growth factor receptor agonist.

21. The use of the oral composition of claim 20 wherein the epidermal growth factor receptor agonist is transforming growth factor alpha, amphiregulin, heparin binding EGF or epiregulin.

22. The use of the oral composition of any of claims 1 to 17 wherein the composition is selected from the group consisting of a solution, suspension, colloid, concentrate, powder, granules, tablets, pressed tablets and capsules.

23. The use of the oral composition of any of claims 1 to 22 wherein the composition comprises from about 1 ng/kg/day to about 10 mg/kg/day of epidermal growth factor, epidermal growth factor receptor agonist or pharmaceutically acceptable salt forms thereof.

24. The use of the oral composition of any of claims 1 to 22 wherein the composition comprises from about 0.1 μg/kg/day to about 1 mg/kg/day of epidermal growth factor, epidermal growth factor receptor agonist or pharmaceutically acceptable salt forms thereof.

25. The use of the oral composition of any of claims 1 to 22 wherein the composition comprises from about 1 μg/kg/day to about 1 mg/kg/day of epidermal growth factor, epidermal growth factor receptor agonist or pharmaceutically acceptable salt forms thereof.

26. The use of the oral composition of any of claims 1 to 22 wherein the composition comprises from about 1 μg/kg/day to about 0.1 mg/kg/day of epidermal growth factor, epidermal growth factor receptor agonist or pharmaceutically acceptable salt forms thereof.

27. An aqueous oral rehydration composition comprising (i) an epidermal growth factor (EGF), an epidermal growth factor receptor agonist, or a pharmaceutically acceptable salt form thereof; (ii) a carbohydrate, and (iii) at least one solute selected from the group consisting of salts and an alternative sodium-coupled nutrient or a source of a sodium-coupled nutrients.

28. The aqueous oral rehydration composition of claim 27 wherein the sodium-coupled nutrient, or the source of sodium-coupled nutrients is selected from the group consisting of amino acids, a source of amino acids, peptides, polypeptides, short-chain fatty acids and a source of non-digestible carbohydrate which can be metabolized to short-chain fatty acids by bacterial fermentation in the gut.

29. The oral rehydration composition of claim 28 comprising an epidermal growth factor.

30. The oral rehydration composition of claim 29 wherein the epidermal growth factor is a human epidermal growth factor.

31. The oral rehydration composition of claim 28 comprising from about 100 picograms to about 1 milligram of epidermal growth factor or epidermal growth factor receptor agonist per milliliter.

32. The oral rehydration composition of claim 31 comprising from about 1 nanogram to about 100 micrograms of epidermal growth factor or epidermal growth factor receptor agonist per milliliter.

33. The oral rehydration composition of claim 32 comprising from about 10 nanograms to about 10 micrograms of epidermal growth factor or epidermal growth factor receptor agonist per milliliter.

34. The oral rehydration composition of any of claims 27 to 33 wherein the rehydration composition is an oral rehydration solution (ORS).

35. The oral rehydration composition of claim 34 wherein the ORS comprises sodium, potassium, chloride, a source of base, and a carbohydrate or a sodium-coupled nutrient or a source of a sodium-coupled nutrients.

36. The ORS of claim 28 wherein (a) sodium is present from about 30 mEq/L to about 95 mEq/L, (b) potassium is present from about 10 mEq/L to about 30 mEq/L, (c) the carbohydrate is present at less than about 5% w/w, (d) the source of base is present from about 10 mEq/L to about 40 mEq/L, and (e) chloride is present from about 30 mEq/L to about 80 mEq/L.

37. The ORS of claim 29 wherein (a) sodium is present from about 30 mEq/L to about 70 mEq/L, (b) potassium is present from about 15 mEq/L to about 25 mEq/L, (c) the carbohydrate is present at less than about 3% w/w, (d) the source of base is present from about 20 mEq/L to about 40 mEq/L, and (e) chloride is present from about 30 mEq/L to about 75 mEq/L.

38. The ORS of claim 30 wherein (a) sodium is present from about 40 mEq/L to about 60 mEq/L, (b) potassium is present from about 15 mEq/L to about 25 mEq/L, (c) the carbohydrate is present from about 2% to about 3% w/w, (d) the source of base is present from about 25 mEq/L to about 35 mEq/L, and (e) chloride is present from about 30 mEq/L to about 70 mEq/L.

39. The ORS of any of claims 35 to 38 wherein (a) the source of base is selected from the group consisting of potassium citrate, sodium citrate, citric acid and mixtures thereof, (b) the carbohydrate is selected from the group consisting of glucose, dextrose, fructooliogosaccharides, fructose polymers, glucose polymers, corn syrup, high fructose corn syrup, sucrose, maltodextrin, rice, rice flour and mixtures thereof, (c) sodium is selected from the group consisting of sodium chloride, sodium citrate, sodium bicarbonate, sodium carbonate, sodium hydroxide, and mixtures thereof, (d) potassium is selected from the group consisting of potassium citrate, potassium chloride, potassium bicarbonate, potassium carbonate, potassium hydroxide and mixtures thereof, and (e) chloride is selected from the group consisting of potassium chloride, sodium chloride, zinc chloride and mixtures thereof.

40. The ORS of any of claims 35 to 39 wherein the sodium-coupled nutrient or source of the sodium-coupled nutrients is selected from the group consisting of amino acids, a source of amino acids, peptides, polypeptides, short-chain fatty acids and a source of non-digestible carbohydrate which can be metabolized to short-chain fatty acids by bacterial fermentation in the gut.

41. The oral rehydration composition of any of claims 27 to 40 further comprising zinc.

42. The oral rehydration composition of any of claims 27 to 40 further comprising glutamine.

43. The oral rehydration composition of any of claims 27 to 40 further comprising an indigestible oligosaccharide.

44. The oral rehydration composition of any of claims 27 to 40 further comprising amidine derivatives.

45. The oral rehydration composition of any of claims 27 to 40 further comprising an additional pharmaceutically active ingredient.

46. The oral rehydration composition of any of claims 27 to 40 further comprising an absorptive component.

47. The oral rehydration composition of any of claims 27 to 40 further comprising glycolipid.

48. The oral rehydration composition of any of claims 27 to 40 wherein the composition is frozen.

49. The oral rehydration composition of any of claims 27 to 40 wherein the composition is in the form of a gel.

50. The oral rehydration composition of any of claims 27 to 49 further comprising a sweetener, flavouring, preservatives, an excipient, a diluent, or an adjuvant.

51. A kit comprising (a) a therapeutic amount of an epidermal growth factor, an epidermal growth factor receptor agonist, or a pharmaceutically acceptable salt form thereof, (b) at least one solute selected from the group consisting of sodium, potassium, chloride, a source of base, a carbohydrate, an alternative sodium-coupled nutrient and a source of sodium-coupled nutrients, and (c) instructions.

52. A method of manufacturing an oral composition comprising providing an epidermal growth factor, and epidermal growth factor receptor agonist, or a pharmaceutically acceptable salt form thereof in the composition and manufacturing the oral composition.

53. The method of claim 52 wherein the epidermal growth factor is a human epidermal growth factor.

54. The method of claim 52 or 53 wherein the composition is an oral rehydration solution (ORS).

55. A method of manufacturing a kit comprising providing (a) a therapeutic amount of an epidermal growth factor, and epidermal growth factor receptor agonist, or a pharmaceutically acceptable salt form thereof, (b) at least one solute selected from the group consisting of sodium, potassium, chloride, a source of base, a carbohydrate, an alternative sodium-coupled nutrient and a source of sodium-coupled nutrients, and (c) instructions.

56. A method of manufacturing a kit comprising providing (a) a therapeutic amount of an epidermal growth factor, (b) at least one solute selected from the group consisting of sodium, potassium, chloride, a source of base, a carbohydrate, sodium-coupled nutrient and a source of sodium-coupled nutrients, and (c) instructions.

57. The method of claim 56 wherein the epidermal growth factor is a human epidermal growth factor.

58. A method for treating diarrhea comprising administering to an animal having diarrhea an effective amount of an epidermal growth factor, an epidermal growth factor receptor agonist, or a pharmaceutically acceptable salt form thereof.

59. A method for reducing the severity of diarrhea comprising administering to an animal having diarrhea an effective amount of an epidermal growth factor, an epidermal growth factor receptor agonist, or a pharmaceutically acceptable salt form thereof.

60. A method for reducing the duration of diarrhea comprising administering to an animal having diarrhea an effective amount of an epidermal growth factor, an epidermal growth factor receptor agonist, or a pharmaceutically acceptable salt form thereof.

61. A method for promoting healing of intestinal damage in an animal having diarrhea comprising administering to an animal having diarrhea an effective amount of an epidermal growth factor, an epidermal growth factor receptor agonist, or a pharmaceutically acceptable salt form thereof.

62. A method for treating dehydration in an animal having diarrhea comprising administering to an animal having dehydration associated with diarrhea an effective amount of an epidermal growth factor, an epidermal growth factor receptor agonist, or a pharmaceutically acceptable salt form thereof.

63. A method for reducing bacterial colonization in an animal having diarrhea comprising administering to an animal having diarrhea an effective amount of an epidermal growth factor, an epidermal growth factor receptor agonist, or a pharmaceutically acceptable salt form thereof.

64. A method for increasing food uptake in an animal having diarrhea comprising administering to an animal having diarrhea an effective amount of an epidermal growth factor, an epidermal growth factor receptor agonist, or a pharmaceutically acceptable salt form thereof.

65. A method for enhancing rehydration in an animal having diarrhea comprising administering to an animal having diarrhea an effective amount of an epidermal growth factor, an epidermal growth factor receptor agonist, or a pharmaceutically acceptable salt form thereof.

66. A method for reducing water content in fecal matter in an animal having diarrhea comprising administering to an animal having diarrhea an effective amount of an epidermal growth factor, an epidermal growth factor receptor agonist, or a pharmaceutically acceptable salt form thereof.

67. A method for increasing villus height in an animal having diarrhea comprising administering to an animal having diarrhea an effective amount of an epidermal growth factor, an epidermal growth factor receptor agonist, or a pharmaceutically acceptable salt form thereof.

68. A method for improving mucosal healing in an animal having diarrhea comprising administering to an animal having diarrhea an effective amount of an epidermal growth factor, an epidermal growth factor receptor agonist, or a pharmaceutically acceptable salt form thereof.

69. A method for enhancing mucosal wet weight in an animal having diarrhea comprising administering to an animal having diarrhea an effective amount of an epidermal growth factor, an epidermal growth factor receptor agonist, or a pharmaceutically acceptable salt form thereof.

70. The method of any one of claims 58 to 69 wherein the diarrhea is infectious malabsorptive diarrhea.

71. The method of any one of claims 58 to 69 wherein the diarrhea is neonatal diarrhea.

72. The method of any one of claims 58 to 69 wherein the diarrhea is secretory diarrhea.

73. The method of any one of claims 58 to 72 comprising administering an effective amount of epidermal growth factor.

74. The method of any one of claims 58 to 72 wherein the epidermal growth factor receptor agonist is transforming growth factor alpha, amphiregulin, heparin binding EGF or epiregulin.

75. The method of any of claims 58 to 72 wherein the epidermal growth factor is a human epidermal growth factor and the animal is a human.

76. The method of any of claims 58 to 74 wherein the animal is selected from the group consisting of human, dog, cat, cow, horse, pig, goat, sheep, rabbits, mink, llamas, alpacas, elk, bison, fish and poultry.

77. A method for treating diarrhea comprising administering to an animal having diarrhea an effective amount of the composition of any of claims 27 to 50.

78. A method for reducing the severity of diarrhea comprising administering to an animal having diarrhea an effective amount of the composition of any of claims 27 to 50.

79. A method for reducing the duration of diarrhea comprising administering to an animal having diarrhea an effective amount of the composition of any of claims 27 to 50.

80. A method for promoting healing of intestinal damage in an animal having diarrhea comprising administering to an animal having diarrhea an effective amount of the composition of any of claims 27 to 50.

81. A method for treating dehydration in an animal having diarrhea comprising administering to an animal having diarrhea an effective amount of the composition of any of claims 27 to 50.

82. A method for reducing bacterial colonization in an animal having diarrhea comprising administering to an animal having diarrhea an effective amount of the composition of any of claims 27 to 50.

83. A method for reducing weight loss in an animal having diarrhea comprising administering to an animal having diarrhea an effective amount of the composition of any of claims 27 to 50.

84. A method for increasing food uptake in an animal having diarrhea comprising administering to an animal having diarrhea an effective amount of the composition of any of claims 27 to 50.

85. A method for enhancing rehydration in an animal having diarrhea comprising administering to an animal having diarrhea an effective amount of the composition of any of claims 27 to 50.

86. A method of reducing water content in fecal matter in an animal having diarrhea comprising administering to an animal having diarrhea an effective amount of the composition of any of claims 27 to 50.

87. A method for increasing villus height in an animal having diarrhea comprising administering to an animal having diarrhea an effective amount of the composition of any of claims 27 to 50.

88. A method for improving mucosal healing in an animal having diarrhea comprising administering to an animal having diarrhea an effective amount of the composition of any of claims 27 to 50.

89. A method for enhancing mucosal wet weight in an animal having diarrhea comprising administering to an animal having diarrhea an effective amount of the composition of any of claims 27 to 50.

90. A method for decreasing bacterial colonization in an animal having diarrhea comprising administering to an animal having diarrhea an effective amount of the composition of any of claims 27 to 50.

91. The method of any one of claims 77 to 90 wherein the diarrhea is infectious malabsorptive diarrhea.

92. The method of any one of claims 77 to 90 wherein the diarrhea is neonatal diarrhea.

93. The method of any one of claims 77 to 90 wherein the diarrhea is secretory diarrhea.

94. A method of treating diarrhea in an animal having a condition selected from the group consisting of recovery from gastrointestinal surgery, gastrointestinal resection, small intestinal transplant, post surgical trauma, short bowel syndrome, burns, oral mucositis, AIDS, inflammatory diseases, Crohn's disease, Ulcerative colitis, celiac disease, necrotizing enterocolitis, gut prematurity, bone marrow transplants, intestinal damage due to chemotherapy or radiation therapy, sepsis, intestinal infections and subjects requiring total parenteral nutrition (TPN) comprising administering the composition of any of claims 20 to 43 to the animal.

95. The method of any one of claims 77 to 94 wherein the composition comprises epidermal growth factor.

96. The method of any one of claims 77 to 94 wherein the epidermal growth factor receptor agonist is transforming growth factor alpha, amphiregulin, heparin binding EGF or epiregulin.

97. The method of any of claims 77 to 94 wherein the epidermal growth factor is a human epidermal growth factor and the animal is a human.

98. The method of any of claims 77 to 94 wherein the animal is selected from the group consisting of human, dog, cat, cow, horse, pig, goat, sheep, rabbits, mink, llamas, alpacas, elk, bison, fish and poultry.

99. A unit dose of epidermal growth factor, epidermal growth factor receptor agonist, or pharmaceutically acceptable salt form thereof for use in a mixture with an oral rehydration solution.

100. The unit dose of claim 99 wherein the dose is epidermal growth factor.

101. The unit dose of claim 100 wherein the epidermal growth factor is human epidermal growth factor.

102. A rehydration composition delivered enterally comprising epidermal growth factor, epidermal growth factor receptor agonist, or pharmaceutically acceptable salt form thereof for use in treating diarrhea, reducing the severity of diarrhea, reducing the duration of diarrhea, promoting intestinal healing, reducing bacterial colonization associated with diarrhea, reducing weight loss in an animal having diarrhea, increasing food uptake in an animal having diarrhea, enhancing rehydration in an animal having diarrhea, reducing water content in fecal matter in an animal having diarrhea, improving mucosal healing in an animal having diarrhea, increasing villus height in an animal having diarrhea, or enhancing mucosal wet weight in an animal having diarrhea.
